SEQUENCE LISTING (114 sequences)

SEQ ID NO: 1 - DNA, 357 bp, Artificial Sequence
  synthetic
caggtgcagctggtggagtctgggggaggcgtggtccagcctgggaagtccctgagactctcctgtgtagcgtctggattcaccttcagttccgatgccatgtactgggtccgccaggctccaggcaaggggctggaatgggtggcagttatttattatgatggaaattatcaatactatgaagactccgttaagggtcgattcaccatctccagagacaattcccagaacacgctggatctgcaaatgaacagcctgagagtcgacgacacggctgtatatttctgtgcgcgtctcaactgggattactggtatctcgatctctggggccgtggcaccctggtcactgtctcctca

SEQ ID NO: 2 - protein, 119 aa, Artificial Sequence
  synthetic
QVQLVESGGGVVQPGKSLRLSCVASGFTFSSDAMYWVRQAPGKGLEWVAVIYYDGNYQYYEDSVKGRFTISRDNSQNTLDLQMNSLRVDDTAVYFCARLNWDYWYLDLWGRGTLVTVSS

SEQ ID NO: 3 - DNA, 24 bp, Artificial Sequence
  synthetic
ggattcaccttcagttccgatgcc

SEQ ID NO: 4 - protein, 8 aa, Artificial Sequence
  synthetic
GFTFSSDA

SEQ ID NO: 5 - DNA, 24 bp, Artificial Sequence
  synthetic
atttattatgatggaaattatcaa

SEQ ID NO: 6 - protein, 8 aa, Artificial Sequence
  synthetic
IYYDGNYQ

SEQ ID NO: 7 - DNA, 36 bp, Artificial Sequence
  synthetic
gcgcgtctcaactgggattactggtatctcgatctc

SEQ ID NO: 8 - protein, 12 aa, Artificial Sequence
  synthetic
ARLNWDYWYLDL

SEQ ID NO: 9 - DNA, 324 bp, Artificial Sequence
  synthetic
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 10 - protein, 108 aa, Artificial Sequence
  synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 11 - DNA, 18 bp, Artificial Sequence
  synthetic
cagagcattagcagctat

SEQ ID NO: 12 - protein, 6 aa, Artificial Sequence
  synthetic
QSISSY

SEQ ID NO: 13 - DNA, 9 bp, Artificial Sequence
  synthetic
gctgcatcc

SEQ ID NO: 14 - protein, 3 aa, Artificial Sequence
  synthetic
AAS

SEQ ID NO: 15 - DNA, 30 bp, Artificial Sequence
  synthetic
caacagagttacagtacccctccgatcacc

SEQ ID NO: 16 - protein, 10 aa, Artificial Sequence
  synthetic
QQSYSTPPIT

SEQ ID NO: 17 - DNA, 357 bp, Artificial Sequence
  synthetic
caggtgcagctggtggagtccgggggaggcgtggtccagcctgggaggtccctgagactctcctgtacagcgtctggattcaccttcagtagttatgccatgtactgggtccgccaggctccaggcaaggggctggagtgggtgtcagttatatactatgatggaagttataaatactatgcagactccgtgaagggccgattcaccatctccagagacaattccaagaacacgctgtatctgcaaatggacagcctgagagccgaggacacggctgtctattactgtgcgagttataactggaactactggtacttcgatttctggggccgtggcaccctggtcactgtctcctca

SEQ ID NO: 18 - protein, 119 aa, Artificial Sequence
  synthetic
QVQLVESGGGVVQPGRSLRLSCTASGFTFSSYAMYWVRQAPGKGLEWVSVIYYDGSYKYYADSVKGRFTISRDNSKNTLYLQMDSLRAEDTAVYYCASYNWNYWYFDFWGRGTLVTVSS

SEQ ID NO: 19 - DNA, 24 bp, Artificial Sequence
  synthetic
ggattcaccttcagtagttatgcc

SEQ ID NO: 20 - protein, 8 aa, Artificial Sequence
  synthetic
GFTFSSYA

SEQ ID NO: 21 - DNA, 24 bp, Artificial Sequence
  synthetic
atatactatgatggaagttataaa

SEQ ID NO: 22 - protein, 8 aa, Artificial Sequence
  synthetic
IYYDGSYK

SEQ ID NO: 23 - DNA, 36 bp, Artificial Sequence
  synthetic
gcgagttataactggaactactggtacttcgatttc

SEQ ID NO: 24 - protein, 12 aa, Artificial Sequence
  synthetic
ASYNWNYWYFDF

SEQ ID NO: 25 - DNA, 357 bp, Artificial Sequence
  synthetic
caggtgcagctggtggagtctgggggaagcgtggtccagcctgggaggtccctgagactctcctgtgcagcgtctggattcaccttcagtacatatgccatgtactgggtccgccagactccaggcaaggggctggagtgggtggctgttttatactctgatggaagtaataaatactatatagactccgtgaagggccgattcaccatctccagagacacttccacgaacactctgtatctgcaaatgagcagcctgcgagccgacgactcggctctatattactgtgcgcgtctcaactgggattactggtacttcgatctctggggccgtggcaccctggtcactgtctcctca

SEQ ID NO: 26 - protein, 119 aa, Artificial Sequence
  synthetic
QVQLVESGGSVVQPGRSLRLSCAASGFTFSTYAMYWVRQTPGKGLEWVAVLYSDGSNKYYIDSVKGRFTISRDTSTNTLYLQMSSLRADDSALYYCARLNWDYWYFDLWGRGTLVTVSS

SEQ ID NO: 27 - DNA, 24 bp, Artificial Sequence
  synthetic
ggattcaccttcagtacatatgcc

SEQ ID NO: 28 - protein, 8 aa, Artificial Sequence
  synthetic
GFTFSTYA

SEQ ID NO: 29 - DNA, 24 bp, Artificial Sequence
  synthetic
ttatactctgatggaagtaataaa

SEQ ID NO: 30 - protein, 8 aa, Artificial Sequence
  synthetic
LYSDGSNK

SEQ ID NO: 31 - DNA, 36 bp, Artificial Sequence
  synthetic
gcgcgtctcaactgggattactggtacttcgatctc

SEQ ID NO: 32 - protein, 12 aa, Artificial Sequence
  synthetic
ARLNWDYWYFDL

SEQ ID NO: 33 - DNA, 357 bp, Artificial Sequence
  synthetic
caggtgcagctggtggagtctgggggaggcgtggtccagcctgggaggtccctgagactctcctgtgaagcgtctggattcagcagcagtgacaatgccatgtactgggtccgccaggctccaggcaaggggctggagtgggtgtcagttatatatcatgatggaagttataaatactatgaagactccgtgaagggccgattcaccatcgccagagacaattccaagaacacgctttatttgcaaatgaacagcctgagagccgaggacacggctgtatattactgtgcgaggtataactggaaccactggtacttcgatgtctggggccgtggcaccctggtcactgtctcctca

SEQ ID NO: 34 - protein, 119 aa, Artificial Sequence
  synthetic
QVQLVESGGGVVQPGRSLRLSCEASGFSSSDNAMYWVRQAPGKGLEWVSVIYHDGSYKYYEDSVKGRFTIARDNSKNTLYLQMNSLRAEDTAVYYCARYNWNHWYFDVWGRGTLVTVSS

SEQ ID NO: 35 - DNA, 24 bp, Artificial Sequence
  synthetic
ggattcagcagcagtgacaatgcc

SEQ ID NO: 36 - protein, 8 aa, Artificial Sequence
  synthetic
GFSSSDNA

SEQ ID NO: 37 - DNA, 24 bp, Artificial Sequence
  synthetic
atatatcatgatggaagttataaa

SEQ ID NO: 38 - protein, 8 aa, Artificial Sequence
  synthetic
IYHDGSYK

SEQ ID NO: 39 - DNA, 36 bp, Artificial Sequence
  synthetic
gcgaggtataactggaaccactggtacttcgatgtc

SEQ ID NO: 40 - protein, 12 aa, Artificial Sequence
  synthetic
ARYNWNHWYFDV

SEQ ID NO: 41 - DNA, 366 bp, Artificial Sequence
  synthetic
caggtgcagctggtggagtctgggggaggcgtggtccagcctgggaggtccctgagactctcctgtgcagcctctggattcaccttcagtacctatggcatgcactgggtccgccaggctccaggcaaggggctggagtgggtgtcagttatatcatatgacgaaagtaataagtactatgcagactccgtgaagggccgattcaccatttctagagacaattccaagaacgcgctgtatttacaaatgaacagcctgagaaatgaggacacggctgtgtattactgtgcgagagatcggccttttggattggttaccggatggttcgacccctggggccagggaaccctggtcaccgtctcctca

SEQ ID NO: 42 - protein, 122 aa, Artificial Sequence
  synthetic
QVQLVESGGGVVQPGRSLRLSCAASGFTFSTYGMHWVRQAPGKGLEWVSVISYDESNKYYADSVKGRFTISRDNSKNALYLQMNSLRNEDTAVYYCARDRPFGLVTGWFDPWGQGTLVTVSS

SEQ ID NO: 43 - DNA, 24 bp, Artificial Sequence
  synthetic
ggattcaccttcagtacctatggc

SEQ ID NO: 44 - protein, 8 aa, Artificial Sequence
  synthetic
GFTFSTYG

SEQ ID NO: 45 - DNA, 24 bp, Artificial Sequence
  synthetic
atatcatatgacgaaagtaataag

SEQ ID NO: 46 - protein, 8 aa, Artificial Sequence
  synthetic
ISYDESNK

SEQ ID NO: 47 - DNA, 45 bp, Artificial Sequence
  synthetic
gcgagagatcggccttttggattggttaccggatggttcgacccc

SEQ ID NO: 48 - protein, 15 aa, Artificial Sequence
  synthetic
ARDRPFGLVTGWFDP

SEQ ID NO: 49 - DNA, 360 bp, Artificial Sequence
  synthetic
caggtgcagctggtggagtctgggggaggcgtggtccagcctgggaggtccctgagactctcctgtgcagcgtctggattcagtttcaatacctatggcatgcactgggtccgccaggctccaggcaaggggctggagtgggtgacaattatatggtatgatggaagtattaaatactatgcagactccgtgaagggccgattcaccatctccagagacaattccaagaacacgctgtatctgcaaatgaacagcctgagagccgaggacacggctgtgtattattgtgcgagaggtggatatagtggctacctctactttgactactggggccagggaaccctggtcaccgtctcctca

SEQ ID NO: 50 - protein, 120 aa, Artificial Sequence
  synthetic
QVQLVESGGGVVQPGRSLRLSCAASGFSFNTYGMHWVRQAPGKGLEWVTIIWYDGSIKYYADSVKGRFTISRDNSKNTLYLQMNSLRAEDTAVYYCARGGYSGYLYFDYWGQGTLVTVSS

SEQ ID NO: 51 - DNA, 24 bp, Artificial Sequence
  synthetic
ggattcagtttcaatacctatggc

SEQ ID NO: 52 - protein, 8 aa, Artificial Sequence
  synthetic
GFSFNTYG

SEQ ID NO: 53 - DNA, 24 bp, Artificial Sequence
  synthetic
atatggtatgatggaagtattaaa

SEQ ID NO: 54 - protein, 8 aa, Artificial Sequence
  synthetic
IWYDGSIK

SEQ ID NO: 55 - DNA, 39 bp, Artificial Sequence
  synthetic
gcgagaggtggatatagtggctacctctactttgactac

SEQ ID NO: 56 - protein, 13 aa, Artificial Sequence
  synthetic
ARGGYSGYLYFDY

SEQ ID NO: 57 - DNA, 372 bp, Artificial Sequence
  synthetic
caggtgcagctgcaggagtcgggcccaggactggtgaagccttcacagaccctgtccctcacctgcactgtctctggtggctccatcagcagcggtggtgactactggagctggatccgccagctcccagggaagggcctggagtggattgggtacatctattacagtgggagcgcctactataatccgtccctcaagagtcgaggtaccatatcaatagacacgtctaagaaccagttctccctgaagctgacctctgtgactgccgcggacacggccgtatatttctgtgtgaaattacgatttttggagtggttcttggggggctggttcggcccctggggccagggaaccctggtcaccgtctcctca

SEQ ID NO: 58 - protein, 124 aa, Artificial Sequence
  synthetic
QVQLQESGPGLVKPSQTLSLTCTVSGGSISSGGDYWSWIRQLPGKGLEWIGYIYYSGSAYYNPSLKSRGTISIDTSKNQFSLKLTSVTAADTAVYFCVKLRFLEWFLGGWFGPWGQGTLVTVSS

SEQ ID NO: 59 - DNA, 30 bp, Artificial Sequence
  synthetic
ggtggctccatcagcagcggtggtgactac

SEQ ID NO: 60 - protein, 10 aa, Artificial Sequence
  synthetic
GGSISSGGDY

SEQ ID NO: 61 - DNA, 21 bp, Artificial Sequence
  synthetic
atctattacagtgggagcgcc

SEQ ID NO: 62 - protein, 7 aa, Artificial Sequence
  synthetic
IYYSGSA

SEQ ID NO: 63 - DNA, 48 bp, Artificial Sequence
  synthetic
gtgaaattacgatttttggagtggttcttggggggctggttcggcccc

SEQ ID NO: 64 - protein, 16 aa, Artificial Sequence
  synthetic
VKLRFLEWFLGGWFGP

SEQ ID NO: 65 - DNA, 351 bp, Artificial Sequence
  synthetic
gaggtgcagctggtggagtctgggggaggcttggtacagccgggggggtccctgagactctcctgtgcagcctctggattcacctttagcaactatggcatgacctgggtccgccaggctccagggaagggcctggaatgggtctcagctattactggtggtggtggtagcacatactactcaaactccgtgaagggccggttcaccatctccagagacaattccaagaacacggtgtatctgcgaatgaacagtgtgagagccgaggacacggccgtatattactgtgcgaaatataagtggaacttcgtggacgactggggccagggaaccacggtcaccgtctcctca

SEQ ID NO: 66 - protein, 117 aa, Artificial Sequence
  synthetic
EVQLVESGGGLVQPGGSLRLSCAASGFTFSNYGMTWVRQAPGKGLEWVSAITGGGGSTYYSNSVKGRFTISRDNSKNTVYLRMNSVRAEDTAVYYCAKYKWNFVDDWGQGTTVTVSS

SEQ ID NO: 67 - DNA, 24 bp, Artificial Sequence
  synthetic
ggattcacctttagcaactatggc

SEQ ID NO: 68 - protein, 8 aa, Artificial Sequence
  synthetic
GFTFSNYG

SEQ ID NO: 69 - DNA, 24 bp, Artificial Sequence
  synthetic
attactggtggtggtggtagcaca

SEQ ID NO: 70 - protein, 8 aa, Artificial Sequence
  synthetic
ITGGGGST

SEQ ID NO: 71 - DNA, 30 bp, Artificial Sequence
  synthetic
gcgaaatataagtggaacttcgtggacgac

SEQ ID NO: 72 - protein, 10 aa, Artificial Sequence
  synthetic
AKYKWNFVDD

SEQ ID NO: 73 - DNA, 360 bp, Artificial Sequence
  synthetic
gaggtgcagctggtggagtctgggggaggcttggtacagcctggggggtccctgagactctcctgtgttgcctctggattcaccttcaataaatacgacatgcactgggtccgccaaactactggaaaaggtctagagtgggtctcaggtattgatactgatggtgacacatactatccaggctccgtgaagggccgattcaccatctccagagaaaatgccgagaactccctgtatcttcaaatgaacggcctgagagtcggggacacggctgtgtattactgtgcaagatggccttggagtggtttctatggtgcttttgatatctggggccaagggacaatggtcaccgtctcttca

SEQ ID NO: 74 - protein, 120 aa, Artificial Sequence
  synthetic
EVQLVESGGGLVQPGGSLRLSCVASGFTFNKYDMHWVRQTTGKGLEWVSGIDTDGDTYYPGSVKGRFTISRENAENSLYLQMNGLRVGDTAVYYCARWPWSGFYGAFDIWGQGTMVTVSS

SEQ ID NO: 75 - DNA, 24 bp, Artificial Sequence
  synthetic
ggattcaccttcaataaatacgac

SEQ ID NO: 76 - protein, 8 aa, Artificial Sequence
  synthetic
GFTFNKYD

SEQ ID NO: 77 - DNA, 21 bp, Artificial Sequence
  synthetic
attgatactgatggtgacaca

SEQ ID NO: 78 - protein, 7 aa, Artificial Sequence
  synthetic
IDTDGDT

SEQ ID NO: 79 - DNA, 42 bp, Artificial Sequence
  synthetic
gcaagatggccttggagtggtttctatggtgcttttgatatc

SEQ ID NO: 80 - protein, 14 aa, Artificial Sequence
  synthetic
ARWPWSGFYGAFDI

SEQ ID NO: 81 - DNA, 366 bp, Artificial Sequence
  synthetic
caggtgcagctgcaggagtcgggcccaggactggtgaagccttcacagaccctgtccctcacctgcactgtctctggtggctccatcagcagtggtaattactactggaactggatccgccaacagccaggagagggcctggagtggattgcttacatctatcacaatggggtcaccaacttcaatccgtccctcaagagtcgacttactatatcagtagacacgtctaagactcagttctccctgaagttgaggtctgtgactgccgcggacacggccgtttattactgtgcgagatcaggcagctggttcgagaactggtacttcgatctctggggccgtggcaccctggtcactgtctcctca

SEQ ID NO: 82 - protein, 122 aa, Artificial Sequence
  synthetic
QVQLQESGPGLVKPSQTLSLTCTVSGGSISSGNYYWNWIRQQPGEGLEWIAYIYHNGVTNFNPSLKSRLTISVDTSKTQFSLKLRSVTAADTAVYYCARSGSWFENWYFDLWGRGTLVTVSS

SEQ ID NO: 83 - DNA, 30 bp, Artificial Sequence
  synthetic
ggtggctccatcagcagtggtaattactac

SEQ ID NO: 84 - protein, 10 aa, Artificial Sequence
  synthetic
GGSISSGNYY

SEQ ID NO: 85 - DNA, 21 bp, Artificial Sequence
  synthetic
atctatcacaatggggtcacc

SEQ ID NO: 86 - protein, 7 aa, Artificial Sequence
  synthetic
IYHNGVT

SEQ ID NO: 87 - DNA, 42 bp, Artificial Sequence
  synthetic
gcgagatcaggcagctggttcgagaactggtacttcgatctc

SEQ ID NO: 88 - protein, 14 aa, Artificial Sequence
  synthetic
ARSGSWFENWYFDL

SEQ ID NO: 89 - DNA, 324 bp, Artificial Sequence
  synthetic
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttggacgttcggccaagggaccaaggtggaaatcaaa

SEQ ID NO: 90 - protein, 108 aa, Artificial Sequence
  synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 91 - DNA, 21 bp, Artificial Sequence
  synthetic
cagagtgttagcagcagctac

SEQ ID NO: 92 - protein, 7 aa, Artificial Sequence
  synthetic
QSVSSSY

SEQ ID NO: 93 - DNA, 9 bp, Artificial Sequence
  synthetic
ggtgcatcc

SEQ ID NO: 94 - protein, 3 aa, Artificial Sequence
  synthetic
GAS

SEQ ID NO: 95 - DNA, 27 bp, Artificial Sequence
  synthetic
cagcagtatggtagctcaccttggacg

SEQ ID NO: 96 - protein, 9 aa, Artificial Sequence
  synthetic
QQYGSSPWT

SEQ ID NO: 97 - DNA, 360 bp, Artificial Sequence
  synthetic
caggtgcagctgcaggagtcgggcccaggactggtgaagccttcggagaccctgtccctcacctgcactgtctctggtggctccatcagtaattcctactggagctggatccggcagcccccagggaagggactggagtggattggatatgtctattcccgtgggaacaccaagtacaacccctccctcacgagtcgagtcaccatgtcatttgacacgtccaagaaccagttctccctgaaactgaggtctgtgaccgccgcagacacggccgtgtattactgtgcgagaagcagcagctggtacgaggactggtacttcgatctctggggccgtggcaccctggtcactgtctcctca

SEQ ID NO: 98 - protein, 120 aa, Artificial Sequence
  synthetic
QVQLQESGPGLVKPSETLSLTCTVSGGSISNSYWSWIRQPPGKGLEWIGYVYSRGNTKYNPSLTSRVTMSFDTSKNQFSLKLRSVTAADTAVYYCARSSSWYEDWYFDLWGRGTLVTVSS

SEQ ID NO: 99 - DNA, 24 bp, Artificial Sequence
  synthetic
ggtggctccatcagtaattcctac

SEQ ID NO: 100 - protein, 8 aa, Artificial Sequence
  synthetic
GGSISNSY

SEQ ID NO: 101 - DNA, 21 bp, Artificial Sequence
  synthetic
gtctattcccgtgggaacacc

SEQ ID NO: 102 - protein, 7 aa, Artificial Sequence
  synthetic
VYSRGNT

SEQ ID NO: 103 - DNA, 42 bp, Artificial Sequence
  synthetic
gcgagaagcagcagctggtacgaggactggtacttcgatctc

SEQ ID NO: 104 - protein, 14 aa, Artificial Sequence
  synthetic
ARSSSWYEDWYFDL

SEQ ID NO: 105 - DNA, 354 bp, Artificial Sequence
  synthetic
caggtgcagctacagcagtggggcgcagggctgtttaagccttcggagaccctgtccctcacctgcgatgtctatggtgggtccttcagaggttattattggagttggatccgccagcccccagggaaggggctggagtggattggggaaatcagttatagtggtttcaccaattacaacccgtccctcaagagtcgagtcatcatatcaatagatacgtccaagaaccagttctccctgaagatgagctctgtgaccgccgcggacacggctgtttattactgtgcgagagttacctatggttatgggacctttgattattggggccagggaaccctggtcaccgtctcctca

SEQ ID NO: 106 - protein, 118 aa, Artificial Sequence
  synthetic
QVQLQQWGAGLFKPSETLSLTCDVYGGSFRGYYWSWIRQPPGKGLEWIGEISYSGFTNYNPSLKSRVIISIDTSKNQFSLKMSSVTAADTAVYYCARVTYGYGTFDYWGQGTLVTVSS

SEQ ID NO: 107 - DNA, 24 bp, Artificial Sequence
  synthetic
ggtgggtccttcagaggttattat

SEQ ID NO: 108 - protein, 8 aa, Artificial Sequence
  synthetic
GGSFRGYY

SEQ ID NO: 109 - DNA, 21 bp, Artificial Sequence
  synthetic
atcagttatagtggtttcacc

SEQ ID NO: 110 - protein, 7 aa, Artificial Sequence
  synthetic
ISYSGFT

SEQ ID NO: 111 - DNA, 36 bp, Artificial Sequence
  synthetic
gcgagagttacctatggttatgggacctttgattat

SEQ ID NO: 112 - protein, 12 aa, Artificial Sequence
  synthetic
ARVTYGYGTFDY

SEQ ID NO: 113 - protein, 446 aa, Artificial Sequence
  synthetic
QVQLVESGGSVVQPGRSLRLSCAASGFTFSTYAMYWVRQTPGKGLEWVAVLYSDGSNKYYIDSVKGRFTISRDTSTNTLYLQMSSLRADDSALYYCARLNWDYWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 114 - protein, 215 aa, Artificial Sequence
  synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0098313 A1
(43) Pub. Date: Mar. 31, 2022

(54) Use of LEPR Agonists for Pain

(71) Applicant: Regeneron Pharmaceuticals, Inc., TARRYTOWN, NY (US)

(72) Inventors: Elif Arioglu Oral, Ann Arbor, MI (US); Baris Akinci, Güzelbahce (TR); Maria Cristina Foss de Freitas, Ypsilanti, MI (US)

(21) Appl. No.: 17/476,078

(22) Filed: Sep. 15, 2021

Related U.S. Application Data
(60) Provisional application No. 63/078,687, filed on Sep. 15, 2020.

(51) Int. Cl. C07K 16/28; A61K 45/06; A61P 25/24

(52) U.S. Cl. CPC C07K 16/2869; A61K 2039/505; A61P 25/24; A61K 45/06

(57) ABSTRACT

The present invention provides methods for reducing pain, the use of opioids and hospitalization in patients suffering with a leptin deficiency or leptin resistance condition such as lipodystrophy. There may be implications of this invention in other forms of chronic pain that involves centralization or hypersensitization of pain by the central nervous system.

This application claims the benefit of U.S. Provisional Patent Application No. 63/078,687, filed Sep. 15, 2020, which is herein incorporated by reference in its entirety.

The sequence listing of the present application is submitted electronically as an ASCII formatted sequence listing with a file name “10823US01_Sequence_Listing_ST25”, creation date of Sep. 15, 2021, and a size of 48 KB. This sequence listing submitted is part of the specification and is herein incorporated by reference in its entirety.

### FIELD OF THE INVENTION

The present invention relates to methods for treating or preventing pain that is associated with lipodystrophy and other leptin deficiency or leptin resistance conditions by administering a LEPR agonist.

### BACKGROUND OF THE INVENTION

Leptin is an adipose tissue hormone that governs energy balance as well as metabolic and neuroendocrine function. In a state of energy deficit, low circulating leptin levels drive adaptive responses, including increasing hunger and energy conservation through modulation of neuroendocrine pathways. Leptin modulates energy and metabolic balance by engaging the leptin receptor (LEPR), a member of the class I cytokine receptor family 3. The LEPR is encoded by a single gene and alternative splicing gives rise to multiple splice isoforms of LEPR that differ at their C-terminal sequence. Of these splice isoforms, LEPR-b is the principal isoform that mediates leptin's effects and is the only isoform to stimulate JAK-STAT signaling.

Leptin deficiency due to genetic loss-of-function mutations in the Lep gene leads to hyperphagia, obesity, insulin resistance, dyslipidemia, and impaired neuroendocrine function in mice that is reversed with leptin treatment. Clinically, the leptin analog, metreleptin, reverses obesity as well as metabolic and reproductive dysfunction in patients with monogenic obesity due to leptin deficiency. Similar to primary leptin deficiency, disease states of secondary hypoleptinemia are associated with glucose and lipid metabolic dysfunction that can be reversed with leptin treatment. Congenital and acquired generalized lipodystrophy syndromes are rare but severe diseases, characterized by a near complete loss of adipose tissue depots. The very low circulating leptin levels in these patients results in a state of hyperphagia, hypertriglyceridemia, hypercholesterolemia, hepatic steatosis, insulin resistance and diabetes.

Pain is associated with some leptin deficiency or leptin resistance conditions such as lipodystrophy (e.g., PLD). For example, some individuals may experience extreme hypertriglyceridemia and chylomicronemia, a condition characterized by the accumulation of fatty droplets called chylomicrons in the plasma. In some cases, this can result in episodes of acute inflammation of the pancreas (pancreatitis). Pancreatitis can be associated with abdominal pain, chills, jaundice, weakness, sweating, vomiting, and weight loss. Patients with panniculitis-associated AGL (acquired generalized lipodystrophy) suffer from sometimes painful inflammation of subcutaneous fat. Fat loss in panniculitis-associated AGL may be localized to a specific part of the body. Ajluni et al., Spectrum of Disease Associated with Partial Lipodystrophy (PL): Lessons from a Trial Cohort, Clin. Endocrinol. (Oxf). 86(5): 698-707 (2017).

There are also features of centralized chronic pain syndrome in patients with lipodystrophy.

Leptin treatment reduces hyperphagia and improves dyslipidemia, hepatic steatosis and glycemic control in Tg-aP2-nSrebp1c mice that develop a near complete loss of adipose depots characteristic of generalized lipodystrophy. These findings translate clinically as metreleptin mitigates metabolic dysfunction in patients with generalized lipodystrophy. At present, metreleptin is approved in the United States to treat the complications of leptin deficiency in patients with generalized lipodystrophy and patients with either generalized or partial lipodystrophy in Japan and the European Union. However, immunogenicity is reported to arise with metreleptin therapy and the potential for neutralizing anti-metreleptin antibodies to cross-react to endogenous leptin is unclear. Although rare, the emergence of this immunogenic response has been associated with reduced efficacy with metreleptin, and metreleptin treatment carries a black box warning.

### SUMMARY OF THE INVENTION

The present invention provides a method for reducing or preventing pain (e.g., abdominal pain (e.g., accompanied by nausea and/or vomiting), liver pain and/or pain due to pancreatitis), anxiety and/or depression associated (e.g., which is leptin deficiency or leptin resistance condition (e.g., partial lipodystrophy)), in a patient, comprising administering, to the patient, an effective amount of LEPR agonist (e.g., REGN4461). In an embodiment of the invention, such pain, anxiety and/or depression is reduced within less than 1, 1, 2, 3, 4, or 5 days of the first administration of the LEPR agonist.

The present invention also provides a method for reducing or maintaining a reduction in: use (e.g., chronic use) of analgesics (e.g., opioids), use of anxiolytics, use of anti-depressants, analgesic (e.g., opioid) seeking behavior, pro re nata or as-needed use of analgesia (e.g., opioids), analgesic (e.g., opioid) overdose, and/or death due to abuse of analgesics (e.g., opioids) in a patient e.g., suffering from a leptin deficiency or leptin resistance condition comprising administering, to the patient, an effective amount of LEPR agonist. In an embodiment of the invention, the use of analgesics, anxiolytics and/or anti-depressants is reduced concomitantly with or prior to the first administration of the LEPR agonist. For example, in an embodiment of the invention, the treating physician has ordered a stop to regular use of analgesics, such as opioids, concomitantly with the initiation of treatment with LEPR agonists (e.g., REGN4461) and such cessation is maintained but for episodic use of analgesics. In an embodiment of the invention, such reduction in use, seeking, overdose or death due to abuse of analgesics refers to that of opioids, but not necessarily non-opioids such as paracetamol.

In an embodiment of the invention, the analgesic is an opioid, a non-opioid, a calcitonin gene-related peptide (CGRP) inhibitor, a cyclooxygenase-2 inhibitor, a gepant, an anti-CGRP monoclonal antibody, a nonsteroidal anti-inflammatory agent, a salicylate, acetaminophen, acetylsalicylic acid, alfentanil, aspirin & citric acid & sodium bicarbonate, bromfenac, celecoxib, choline salicylate & magnesium salicylate, codeine, concentrate of poppy straw, dextromoramide, dextropropoxyphene, diclofenac, diclofenac & misoprostol, diflunisal, diflunisal, dihydrocodeine, dihydroetorphine, diphenoxylate, eptinezumab, erenumab, esomeprazole & naproxen, ethylmorphine, etodolac, etorphine, famotidine & ibuprofen, fenoprofen, fentanyl, flurbiprofen, fremanezumab, gabapentin, galcanezumab, heroin, hydrocodone, hydromorphone, ibuprofen, indomethacin, ketamin, ketobemidone, ketoprofen, ketorolac, levorphanol, magnesium salicylate, meclofenamat, mefenamic acid, meloxicam, methadone, methadone, morphine, morphine-n-oxide, nabumetone, naproxen, nicomorphine, norcodeine, opium, oripavine, oxaprozin, oxycodone, oxymorphone, pethidine, pethidine intermediate, phenylbutazone, pholcodine, piritramide, piroxicam, remifentanil, rimegepant sulfate, salsalate, sufentanil, sulindac, thebaine or tilidine, tolmetin, ubrogepant or valdecoxib.

In an embodiment of the invention, the anxiolytic is a benzodiazepine, a tricyclic antidepressant, alprazolam, alprazolam, an agonist of melatonin receptor, an anesthetic, an antihistamine, an SNRI, an SSRI, buspirone, clonazepam, diazepam, estazolam, eszopiclone, flurazepam, lorazepam, quazepam, temazepam, triazolam, zaleplon, zolpidem or zopiclone.

In an embodiment of the invention, the anti-depressant is a monoamine oxidase inhibitor, a selective serotonin reuptake inhibitors (SSRI), a serotonin and norepinephrine reuptake inhibitors (SNRI), a tricyclic antidepressant, amitriptyline, an atypical antidepressant, bupropion, citalopram, desipramine, desvenlafaxine and levomilnacipran, doxepin, duloxetine, escitalopram, fluoxetine, imipramine, isocarboxazid, mirtazapine, nortriptyline, paroxetine, phenelzine, selegiline, sertraline, tranylcypromine, trazodone, venlafaxine, vilazodone or vortioxetine.

The present invention provides a method of reducing hospitalization of a patient suffering from a leptin deficiency or leptin resistance condition due to pain (e.g., abdominal pain (e.g., accompanied by nausea and/or vomiting), liver pain and/or pain due to pancreatitis), anxiety and/or depression comprising administering, to the patient, an effective amount of LEPR agonist.

In an embodiment of the invention, the leptin deficiency or leptin resistance condition is monogenic obesity, obesity, metabolic syndrome, diet-induced food craving, functional hypothalamic amenorrhea, type 1 diabetes, type 2 diabetes, insulin resistance, possession of neutralizing anti-leptin autoantibodies, severe insulin resistance including severe insulin resistance due to mutation in insulin receptor, severe insulin resistance not caused by mutation in the insulin receptor, severe insulin resistance caused by a mutation in downstream signaling pathways or induced by other causes, non-alcoholic and alcoholic fatty liver diseases, Alzheimer's disease, leptin deficiency, leptin resistance, a lipodystrophy (e.g., congenital generalized lipodystrophy, acquired generalized lipodystrophy, familial partial lipodystrophy, acquired partial lipodystrophy, centrifugal abdominal lipodystrophy, lipoatrophia annularis, localized lipodystrophy, and HIV-associated lipodystrophy), Leprechaunism/Donohue syndrome or Rabson-Mendenhall syndrome.

In an embodiment of the invention, the LEPR agonist is an isolated antibody or antigen-binding fragment that binds specifically to LEPR, for example, comprising: (i) a light chain variable region that comprises the LCDR1, LCDR2 and LCDR3 of a light chain variable region that comprises the amino acid sequence set forth in SEQ ID NO: 10; and a heavy chain variable region that comprises the HCDR1, HCDR2 and HCDR3 of a heavy chain variable region that comprises the amino acid sequence set forth in SEQ ID NO: 2; (ii) a light chain variable region that comprises the LCDR1, LCDR2 and LCDR3 of a light chain variable region that comprises the amino acid sequence set forth in SEQ ID NO: 10; and a heavy chain variable region that comprises the HCDR1, HCDR2 and HCDR3 of a heavy chain variable region that comprises the amino acid sequence set forth in SEQ ID NO: 18; (iii) a light chain variable region that comprises the LCDR1, LCDR2 and LCDR3 of a light chain variable region that comprises the amino acid sequence set forth in SEQ ID NO: 10; and a heavy chain variable region that comprises the HCDR1, HCDR2 and HCDR3 of a heavy chain variable region that comprises the amino acid sequence set forth in SEQ ID NO: 26; (iv) a light chain variable region that comprises the LCDR1, LCDR2 and LCDR3 of a light chain variable region that comprises the amino acid sequence set forth in SEQ ID NO: 10; and a heavy chain variable region that comprises the HCDR1, HCDR2 and HCDR3 of a heavy chain variable region that comprises the amino acid sequence set forth in SEQ ID NO: 34; (v) a light chain variable region that comprises the LCDR1, LCDR2 and LCDR3 of a light chain variable region that comprises the amino acid sequence set forth in SEQ ID NO: 10; and a heavy chain variable region that comprises the HCDR1, HCDR2 and HCDR3 of a heavy chain variable region that comprises the amino acid sequence set forth in SEQ ID NO: 42; (vi) a light chain variable region that comprises the LCDR1, LCDR2 and LCDR3 of a light chain variable region that comprises the amino acid sequence set forth in SEQ ID NO: 10; and a heavy chain variable region that comprises the HCDR1, HCDR2 and HCDR3 of a heavy chain variable region that comprises the amino acid sequence set forth in SEQ ID NO: 50; (vii) a light chain variable region that comprises the LCDR1, LCDR2 and LCDR3 of a light chain variable region that comprises the amino acid sequence set forth in SEQ ID NO: 10; and a heavy chain variable region that comprises the HCDR1, HCDR2 and HCDR3 of a heavy chain variable region that comprises the amino acid sequence set forth in SEQ ID NO: 58; (viii) a light chain variable region that comprises the LCDR1, LCDR2 and LCDR3 of a light chain variable region that comprises the amino acid sequence set forth in SEQ ID NO: 10; and a heavy chain variable region that comprises the HCDR1, HCDR2 and HCDR3 of a heavy chain variable region that comprises the amino acid sequence set forth in SEQ ID NO: 66; (ix) a light chain variable region that comprises the LCDR1, LCDR2 and LCDR3 of a light chain variable region that comprises the amino acid sequence set forth in SEQ ID NO: 10; and a heavy chain variable region that comprises the HCDR1, HCDR2 and HCDR3 of a heavy chain variable region that comprises the amino acid sequence set forth in SEQ ID NO: 74; (x) a light chain variable region that comprises the LCDR1, LCDR2 and LCDR3 of a light chain variable region that comprises the amino acid sequence set forth in SEQ ID NO: 90; and a heavy chain variable region that comprises the HCDR1, HCDR2 and HCDR3 of a heavy chain variable region that comprises the amino acid sequence set forth in SEQ ID NO: 82; (xi) a light chain variable region that comprises the LCDR1, LCDR2 and LCDR3 of a light chain variable region that comprises the amino acid sequence set forth in SEQ ID NO: 90; and a heavy chain variable region that comprises the HCDR1, HCDR2 and HCDR3 of a heavy chain variable region that comprises the amino acid sequence set forth in SEQ ID NO: 98; or (xii) a light chain variable region that comprises the LCDR1, LCDR2 and LCDR3 of a light chain variable region that comprises the amino acid sequence set forth in SEQ ID NO: 90; and a heavy chain variable region that comprises the HCDR1, HCDR2 and HCDR3 of a heavy chain variable region that comprises the amino acid sequence set forth in SEQ ID NO: 106. In an embodiment of the invention, the isolated antibody or antigen-binding fragment that binds specifically to LEPR comprises: (i) a light chain variable region that comprises the amino acid sequence set forth in SEQ ID NO: 10; and a heavy chain variable region that comprises the amino acid sequence set forth in SEQ ID NO: 2; (ii) a light chain variable region that comprises the amino acid sequence set forth in SEQ ID NO: 10; and a heavy chain variable region that comprises the amino acid sequence set forth in SEQ ID NO: 18; (iii) a light chain variable region that comprises the amino acid sequence set forth in SEQ ID NO: 10; and heavy chain variable region that comprises the amino acid sequence set forth in SEQ ID NO: 26; (iv) a light chain variable region that comprises the amino acid sequence set forth in SEQ ID NO: 10; and a heavy chain variable region that comprises the amino acid sequence set forth in SEQ ID NO: 34; (v) a light chain variable region that comprises the amino acid sequence set forth in SEQ ID NO: 10; and a heavy chain variable region that comprises the amino acid sequence set forth in SEQ ID NO: 42; (vi) a light chain variable region that comprises the amino acid sequence set forth in SEQ ID NO: 10; and a heavy chain variable region that comprises the amino acid sequence set forth in SEQ ID NO: 50; (vii) a light chain variable region that comprises the amino acid sequence set forth in SEQ ID NO: 10; and a heavy chain variable region that comprises the amino acid sequence set forth in SEQ ID NO: 58; (viii) a light chain variable region that comprises the amino acid sequence set forth in SEQ ID NO: 10; and a heavy chain variable region that comprises the amino acid sequence set forth in SEQ ID NO: 66; (ix) a light chain variable region that comprises the amino acid sequence set forth in SEQ ID NO: 10; and a heavy chain variable region that comprises the amino acid sequence set forth in SEQ ID NO: 74; (x) a light chain variable region that comprises the amino acid sequence set forth in SEQ ID NO: 90; and a heavy chain variable region that comprises the amino acid sequence set forth in SEQ ID NO: 82; (xi) a light chain variable region that comprises the amino acid sequence set forth in SEQ ID NO: 90; and a heavy chain variable region that comprises the amino acid sequence set forth in SEQ ID NO: 98; or (xii) a light chain variable region that comprises the amino acid sequence set forth in SEQ ID NO: 90; and a heavy chain variable region that comprises the amino acid sequence set forth in SEQ ID NO: 106.

In an embodiment of the invention, the effective amount of LEPR agonist is one or more intravenous doses of about 5 mg/kg and one or more subcutaneous doses of about 250-300 mg once weekly thereafter.

In an embodiment of the invention, the patient is also administered a further therapeutic agent, for example, human leptin, metreleptin, a PCSK9 inhibitor, an anti-PCSK9 antagonist antibody, alirocumab, evolocumab, bococizumab, lodelcizumab, ralpancizumab, an HMG-CoA reductase inhibitor, atorvastatin, rosuvastatin, cerivastatin, pitavastatin, fluvastatin, simvastatin, lovastatin, pravastatin, ezetimibe, insulin, an insulin variant, an insulin secretagogue, metformin, a sulfonylurea, a sodium glucose cotransporter 2 (SGLT2) inhibitor, dapaglifozin, canaglifozin, empagliflozin, a selective agonist of the MC₄ receptor, setmelanotide, a GLP-1 agonist or analogue, extendin-4, exenatide, liraglutide, lixisenatide, albiglutide, dulaglutide, a glucagon (GCG) inhibitor, an anti-GCG antibody, a glucagon receptor (GCGR) inhibitor, an anti-GCGR antibody, a small molecule GCGR antagonist, a GCGR-specific antisense oligonucleotide, an anti-GCGR aptamer, an angiopoietin-like protein (ANGPTL) inhibitor, an anti-ANGPTL3 antibody, an anti-ANGPTL4 antibody, an anti-ANGPTL8 antibody, phentermine, orlistat, topiramate, bupropion, topiramate and phentermine, bupropion and naltrexone, bupropion and zonisamide, pramlintide and metrelepin, lorcaserin, cetilistat, tesofensine or velneperit.

### BRIEF DESCRIPTION OF THE FIGURES

FIG. 1. A summary of the course of a partial lipodystrophy (PLD) patient's disease over time (age in years) and her metabolic parameters during different therapeutic interventions.

FIG. 2. Physical appearance of the PLD patient over time wherein a reduction in the patient's liver size from baseline was evident by physical examination of liver span.

FIG. 3. Resting energy expenditure (REE) of the PLD patient over time. The REE represents roughly 60% of total energy expenditure, and decreased from 2599 kcal at baseline to 1799 kcal.

FIG. 4. Respiratory quotient (RQ) of the PLD patient over time. RQ decreased from 0.92 at baseline to 0.81 at week 12 and 0.82 at week 25.

FIG. 5. The PHQ-9 score of the PLD patient over time. A higher score indicates more depressive symptoms.

FIG. 6. SF-36 scores of the PLD patient over time. A higher score corresponds to an improvement in the indicated item.

FIG. 7. Summary of major pain episodes and medication usage by patient. Reflects some of the data set forth in Table 1-3. PRN oxycodone use (prior to the initiation of REGN4461) was at least 2 to 3 doses for at least 3 days of the week for at least 12 months. Double curved lines indicates that the timeline is not to scale.

### DETAILED DESCRIPTION OF THE INVENTION

The present invention includes methods for treating pain that is associated with lipodystrophy. Patients suffering from lipodystrophy conditions typically suffer from various types of physical pain that are caused by physical changes attendant to their condition, e.g., pancreatitis, enlarged liver. While alleviating these physical changes can, in turn, reduce the pain they cause, the anti-LEPR antibodies set forth herein have been observed to result in a very rapid reduction in physical pain. Unexpectedly, this reduction was observed to occur before any significant physical change occurred. LEPR pathways in the brain may be implicated in wider chronic pain syndromes in as much as this pathway may be involved with pain perception by the brain; thus, making the anti-LEPR antibodies discussed herein useful for modulating such a pathway and/or treating or preventing such conditions.

### LEPR Agonists

The present invention includes methods of using LEPR agonists for the treatment of pain anxiety and/or depression, for example, which is associated with a leptin deficiency or leptin resistance condition (e.g., partial lipodystrophy). A LEPR agonist refers to a molecule or substance that activates LEPR signaling (the stimulation of an intracellular effect that normally results from the interaction of leptin with LEPR in cells that express LEPR). In certain embodiments, the activation of LEPR signaling refers to transcriptional activation of STAT3, which can be detected using any method that can measure or identify, directly or indirectly, STAT3 activity, e.g., using a labeled version of STAT3 expressed in a reporter cell line. For example, the present invention includes the use of LEPR agonists that activate LEPR signaling in a cell-based reporter assay, e.g., using a cell based assay format as defined in Example 7 as set forth in WO2017/66204, or a substantially similar assay. Cell-based reporter assays that detect LEPR activation, such as the assay set forth in Example 7 as set forth in WO2017/66204, can produce a detectable signal that may be expressed in terms of an EC₅₀ value (i.e., the agonist concentration required to produce half-maximal signaling) and/or a percentage of the maximal signaling observed in the presence of leptin. In certain exemplary embodiments of the present invention, LEPR agonists are provided that activate LEPR signaling with an EC₅₀ value of less than about 12.0 nM in a cell-based reporter assay, e.g., using an assay format as defined in Example 7 as set forth in WO2017/66204, or a substantially similar assay. In certain exemplary embodiments of the present invention, LEPR agonists activate LEPR signaling with maximum percent activation relative to leptin signaling of greater than about 65% in a cell-based reporter assay, e.g., using an assay format as defined in Example 7 as set forth in WO2017/66204, or a substantially similar assay. LEPR agonists include antibodies and antigen-binding fragments thereof that bind specifically to LEPR as well as small molecules.

The term “antibody”, as used herein, refers to immunoglobulin molecules comprising four polypeptide chains, two heavy chains (HCs) and two light chains (LCs), inter-connected by disulfide bonds (e.g., an IgG)-REGN4461. In an embodiment of the invention, each antibody heavy chain (HC) comprises a heavy chain variable region (“HCVR” or “V_{H}”) (e.g., SEQ ID NO: 2, 18, 26, 34, 42, 50, 58, 66, 74, 82, 98 or 106 or a variant thereof) and a heavy chain constant region; and each antibody light chain (LC) comprises a light chain variable region (“LCVR or “V_{L}”) (e.g., SEQ ID NO: 10 or 90 a variant thereof) and a light chain constant region (CL). The V_{H} and V_{L} regions can be further subdivided into regions of hypervariability, termed complementarity determining regions (CDR), interspersed with regions that are more conserved, termed framework regions (FR). Each V_{H} and V_{L} comprises three CDRs.

In an embodiment of the invention, the assignment of amino acids to each framework or CDR domain is in accordance with the definitions of Kabat, Chothia or Abm: See e.g., Sequences of Proteins of Immunological Interest, Kabat, et al.; National Institutes of Health, Bethesda, Md.; 5^{th} ed.; NIH Publ. No. 91-3242 (1991); Kabat, The Structural Basis for Antibody Complementary, Adv. Prot. Chem. 32:1-75 (1978); Kabat, et al., Unusual distributions of amino acids in complementarity-determining (hypervariable) segments of heavy and light chains of immunoglobulins and their possible roles in specificity of antibody-combining sites, J. Biol. Chem. 252:6609-6616 (1977); Chothia, et al., Canonical structures for the hypervariable regions of immunoglobulins, J. Mol. Biol. 196:901-917 (1987) or Chothia, et al., Conformations of immunoglobulin hypervariable regions, Nature 342:877-883 (1989). Thus, the present invention includes antibodies and antigen-binding fragments including the CDRs of a V_{H} and the CDRs of a V_{L}, which V_{H} and V_{L} comprise amino acid sequences as set forth herein (or a variant thereof), wherein the CDRs are as defined according to Kabat and/or Chothia. See also AI-Lazikani et al., J. Mol. Biol., Standard conformations for the canonical structures of immunoglobulins. 273: 927-48 (1997); Martin, et al., Modeling antibody hypervariable loops: a combined algorithm, Proc. Natl. Acad. Sci. USA 86: 9268-9272 (1989); Martin et al., Molecular modeling of antibody combining sites, Methods Enzymol., 203: 121-153 (1991); Pedersen et al., Antibody modeling: Beyond homology, Immunomethods 1(2): 126-136 (1992); and Rees et al., In Sternberg M. J. E. (ed.), Protein Structure Prediction. Oxford University Press, Oxford, 141-172. (1996)).

In an embodiment of the invention, an anti-LEPR antibody or antigen-binding fragment comprises a heavy chain constant domain, e.g., of the type IgA (e.g., IgA1 or IgA2), IgD, IgE, IgG (e.g., IgG1, IgG2, IgG3 and IgG4 (e.g., comprising a S228P and/or S108P mutation)) or IgM. In an embodiment of the invention, an antigen-binding protein, e.g., antibody or antigen-binding fragment, comprises a light chain constant domain, e.g., of the type kappa or lambda or a variant thereof, e.g., as set forth herein. The present invention includes antibodies and antigen-binding fragments comprising the variable domains set forth herein which are linked to a heavy and/or light chain constant domain, e.g., as set forth herein.

“Isolated” antibodies or antigen-binding fragments thereof, polypeptides, polynucleotides and vectors, etc., are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules (e.g., minor or insignificant amounts of impurity may remain) or to an absence of water, buffers, or salts or to components of a pharmaceutical formulation that includes the antibodies or antigen-binding fragments.

Non-limiting examples of anti-LEPR antigen-binding fragments include: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An anti-LEPR antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}—V_{H}, V_{H}—V_{L} or V_{L}—V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}—C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-CL; (viii) VL-C_{H}1; (ix) V_{L}-C_{H}2; (X) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

Antibodies and antigen-binding fragments that bind specifically to LEPR may be referred to as “anti-LEPR”. An antibody or fragment binds specifically to LEPR if it binds monomeric human LEPR wherein such binding is characterized by a K_{D} of less than about 150 nM as measured by surface plasmon resonance at 25° C. or 37° C.

TABLE A-1
Nucleotide Sequences Encoding Anti-LEPR Agonist
Antibodies and Antigen-Binding Fragments thereof

| Antibody/Fragment | SEQ ID NOs: | | | | | | | |
|---|---|---|---|---|---|---|---|---|
| Designation | HCVR | HCDR 1 | HCDR 2 | HCDR 3 | LCVR | LCDR 1 | LCDR 2 | LCDR 3 |
| H4H16650P2 | 1 | 3 | 5 | 7 | 9 | 11 | 13 | 15 |
| H4H16679P2 | 17 | 19 | 21 | 23 | 9 | 11 | 13 | 15 |
| H4H17319P2 | 25 | 27 | 29 | 31 | 9 | 11 | 13 | 15 |
| H4H17321P2 | 33 | 35 | 37 | 39 | 9 | 11 | 13 | 15 |
| H4H18417P2 | 41 | 43 | 45 | 47 | 9 | 11 | 13 | 15 |
| H4H18438P2 | 49 | 51 | 53 | 55 | 9 | 11 | 13 | 15 |
| H4H18445P2 | 57 | 59 | 61 | 63 | 9 | 11 | 13 | 15 |
| H4H18446P2 | 65 | 67 | 69 | 71 | 9 | 11 | 13 | 15 |
| H4H18449P2 | 73 | 75 | 77 | 79 | 9 | 11 | 13 | 15 |
| H4H18482P2 | 81 | 83 | 85 | 87 | 89 | 91 | 93 | 95 |
| H4H18487P2 | 97 | 99 | 101 | 103 | 89 | 91 | 93 | 95 |
| H4H18492P2 | 105 | 107 | 109 | 111 | 89 | 91 | 93 | 95 |

TABLE A-2
Amino Acid Sequences of Anti-LEPR Agonist
Antibodies and Antigen-Binding Fragments thereof

| Antibody/Fragment | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H4H16650P2 | 2 | 4 | 6 | 8 | 10 | 12 | 14 | 16 |
| H4H16679P2 | 18 | 20 | 22 | 24 | 10 | 12 | 14 | 16 |
| H4H17319P2 | 26 | 28 | 30 | 32 | 10 | 12 | 14 | 16 |
| H4H17321P2 | 34 | 36 | 38 | 40 | 10 | 12 | 14 | 16 |
| H4H18417P2 | 42 | 44 | 46 | 48 | 10 | 12 | 14 | 16 |
| H4H18438P2 | 50 | 52 | 54 | 56 | 10 | 12 | 14 | 16 |
| H4H18445P2 | 58 | 60 | 62 | 64 | 10 | 12 | 14 | 16 |
| H4H18446P2 | 66 | 68 | 70 | 72 | 10 | 12 | 14 | 16 |
| H4H18449P2 | 74 | 76 | 78 | 80 | 10 | 12 | 14 | 16 |
| H4H18482P2 | 82 | 84 | 86 | 88 | 90 | 92 | 94 | 96 |
| H4H18487P2 | 98 | 100 | 102 | 104 | 90 | 92 | 94 | 96 |
| H4H18492P2 | 106 | 108 | 110 | 112 | 90 | 92 | 94 | 96 |

See International Patent Application No. PCT/US2016/056465; publication no. W02017/066204.

Heavy chain and light chain variable regions of exemplary anti-LEPR agonist antibodies and antigen-binding fragments thereof are set forth below.

H4H16650P2
HCVR(V_{H})
(SEQ ID NO: 2)
QVQLVESGGGVVQPGKSLRLSCVASGFTFSSDAMYWVRQAPGKGLEWVAVIYYDGNYQYY
EDSVKGRFTISRDNSQNTLDLQMNSLRVDDTAVYFCARLNWDYWYLDLWGRGTLVTVSS
LCVR(V_{L})
(SEQ ID NO: 10)
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIK
H4H16679P2
HCVR
(SEQ ID NO: 18)
QVQLVESGGGWQPGRSLRLSCTASGFTFSSYAMYWVRQAPGKGLEWVSVIYYDGSYKYYA
DSVKGRFTISRDNSKNTLYLQMDSLRAEDTAVYYCASYNWNYWYFDFWGRGTLVTVSS
LCVR
(SEQ ID NO: 10)
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIK
H4H17319P2
HCVR
(SEQ ID NO: 26)
QVQLVESGGSVVQPGRSLRLSCAASGFTFSTYAMYWVRQTPGKGLEWVAVLYSDGSNKYY
IDSVKGRFTISRDTSTNTLYLQMSSLRADDSALYYCARLNWDYWYFDLWGRGTLVTVSS
LCVR
(SEQ ID NO: 10)
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIK
H4H17321P2
HCVR
(SEQ ID NO: 34)
QVQLVESGGGVVQPGRSLRLSCEASGFSSSDNAMYWVRQAPGKGLEWVSVIYHDGSYKYY
EDSVKGRFTIARDNSKNTLYLQMNSLRAEDTAVYYCARYNWNHWYFDVWGRGTLVTVSS
LCVR
(SEQ ID NO: 10)
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIK
H4H18417P2
HCVR
(SEQ ID NO: 42)
QVQLVESGGGVVQPGRSLRLSCAASGFTFSTYGMHWVRQAPGKGLEWVSVISYDESNKYY
ADSVKGRFTISRDNSKNALYLQMNSLRNEDTAVYYCARDRPFGLVTGWFDPWGQGTLVTV
SS
LCVR
(SEQ ID NO: 10)
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIK
H4H18438P2
HCVR
(SEQ ID NO: 50)
QVQLVESGGGVVQPGRSLRLSCAASGFSFNTYGMHWVRQAPGKGLEWVTIIWYDGSIKYY
ADSVKGRFTISRDNSKNTLYLQMNSLRAEDTAVYYCARGGYSGYLYFDYWGQGTLVTVSS
LCVR
(SEQ ID NO: 10)
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIK
H4H18445P2
HCVR
(SEQ ID NO: 58)
QVQLQESGPGLVKPSQTLSLTCTVSGGSISSGGDYWSWIRQLPGKGLEWIGYIYYSGSAY
YNPSLKSRGTISIDTSKNQFSLKLTSVTAADTAVYFCVKLRFLEWFLGGWFGPWGQGTLV
TVSS
LCVR
(SEQ ID NO: 10)
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIK
H4H18446P2
HCVR
(SEQ ID NO: 66)
EVQLVESGGGLVQPGGSLRLSCAASGFTFSNYGMTWVRQAPGKGLEWVSAITGGGGSTYY
SNSVKGRFTISRDNSKNTVYLRMNSVRAEDTAVYYCAKYKWNFVDDWGQGTTVTVSS
LCVR
(SEQ ID NO: 10)
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIK
H4H18449P2
HCVR
(SEQ ID NO: 74)
EVQLVESGGGLVQPGGSLRLSCVASGFTFNKYDMHWVRQTTGKGLEWVSGIDTDGDTYYP
GSVKGRFTISRENAENSLYLQMNGLRVGDTAVYYCARWPWSGFYGAFDIWGQGTMVTVSS
LCVR
(SEQ ID NO: 10)
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIK
H4H18482P2
HCVR
(SEQ ID NO: 82)
QVQLQESGPGLVKPSQTLSLTCTVSGGSISSGNYYWNWIRQQPGEGLEWIAYIYHNGVTN
FNPSLKSRLTISVDTSKTQFSLKLRSVTAADTAVYYCARSGSWFENWYFDLWGRGTLVTV
SS
LCVR
(SEQ ID NO: 90)
EIVLTQSPGTLSLSPGERATLSCRASQSVSSSYLAWYQQKPGQAPRLLIYGASSRATGIP
DRFSGSGSGTDFTLTISRLEPEDFAVYYCQQYGSSPWTFGQGTKVEIK
H4H18487P2
HCVR
(SEQ ID NO: 98)
QVQLQESGPGLVKPSETLSLTCTVSGGSISNSYWSWIRQPPGKGLEWIGYVYSRGNTKYN
PSLTSRVTMSFDTSKNQFSLKLRSVTAADTAVYYCARSSSWYEDWYFDLWGRGTLVTVSS
LCVR
(SEQ ID NO: 90)
EIVLTQSPGTLSLSPGERATLSCRASQSVSSSYLAWYQQKPGQAPRLLIYGASSRATGIP
DRFSGSGSGTDFTLTISRLEPEDFAVYYCQQYGSSPWTFGQGTKVEIK
H4H18492P2
HCVR
(SEQ ID NO: 106)
QVQLQQWGAGLFKPSETLSLTCDVYGGSFRGYYWSWIRQPPGKGLEWIGEISYSGFTNYN
PSLKSRVIISIDTSKNQFSLKMSSVTAADTAVYYCARVTYGYGTFDYWGQGTLVTVSS
LCVR
(SEQ ID NO: 90)
EIVLTQSPGTLSLSPGERATLSCRASQSVSSSYLAWYQQKPGQAPRLLIYGASSRATGIP
DRFSGSGSGTDFTLTISRLEPEDFAVYYCQQYGSSPWTFGQGTKVEIK

In an embodiment of the invention, the LEPR agonist is mibavademab. WHO Drug Information, Vol. 34, No. 4, 2020; Proposed INN: List 124.

“H4H16650P2”, “H4H16679P2”, “H4H17319P2”, “H4H17321 P2”, “H4H18417P2”, “H4H18438P2”, “H4H18445P2”, “H4H18446P2”, “H4H18449P2”, “H4H18482P2”, “H4H18487P2” and “H4H18492P2” refer to anti-LEPR agonist antibodies and antigen-binding fragments thereof (including multi-specific antigen-binding proteins), comprising the immunoglobulin heavy chain or variable region thereof (V_{H}) of SEQ ID NO: 2, 18, 26, 34, 42, 50, 58, 66, 74, 82, 98 or 106 (or a variant thereof); and the immunoglobulin light chain or variable region thereof (V_{L}) of 10 or 90 (or a variant thereof), as set forth above in Table A; or that comprise a heavy chain or V_{H} that comprises the CDRs thereof (CDR-H1 (or a variant thereof), CDR-H2 (or a variant thereof) and CDR-H3 (or a variant thereof)) and/or a light chain or V_{L} that comprises the CDRs thereof (CDR-L1 (or a variant thereof), CDR-L2 (or a variant thereof) and CDR-L3 (or a variant thereof)), e.g., wherein the immunoglobulin chains, variable regions and/or CDRs comprise the specific amino acid sequences described below. In an embodiment of the invention, the V_{H} is linked to an IgG constant heavy chain domain (e.g., IgG1 or IgG4 or a variant thereof) and/or the V_{L} is linked to a lambda or kappa constant light chain domain (or a variant thereof).

A “variant” of a polypeptide, such as an immunoglobulin chain (e.g., of H4H16650P2, H4H16679P2, H4H17319P2, H4H17321P2, H4H18417P2, H4H18438P2, H4H18445P2, H4H18446P2, H4H18449P2, H4H18482P2, H4H18487P2 and H4H18492P2 V_{H}, V_{L}, HC or LC), includes a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein (e.g., any of SEQ ID NOs: 2, 10, 18, 26, 34, 42, 50, 58, 66, 74, 82, 90, 98 or 106); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment). A variant may include a polypeptide identical or similar to a referenced amino acid sequence that is set forth herein (e.g., any of SEQ ID NOs: 2, 10, 18, 26, 34, 42, 50, 58, 66, 74, 82, 90, 98 or 106), but having 1, 2, 3, 4, 5, 6, 7, 8, 9, 10 or more mutations. The mutations can include point mutations which are conservative or non-conservative amino acid, substitutions, insertions or deletions.

The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

Exemplary anti-LEPR agonist antibodies and antigen-binding fragments thereof which are used in methods of the present invention are listed in Table A herein. Table A sets forth the amino acid sequence identifiers of the heavy chain variable regions (HCVRs), light chain variable regions (LCVRs), heavy chain complementarity determining regions (HCDR1, HCDR2 and HCDR3), and light chain complementarity determining regions (LCDR1, LCDR2 and LCDR3) of the exemplary anti-LEPR agonist antibodies and antigen-binding fragments.

The present invention provides methods of using anti-LEPR agonist antibodies and antigen-binding fragments thereof comprising an HCVR comprising an amino acid sequence selected from any of the HCVR amino acid sequences listed in Table A; or a variant thereof.

The present invention also provides methods of using anti-LEPR agonist antibodies and antigen-binding fragments thereof, comprising an LCVR comprising an amino acid sequence selected from any of the LCVR amino acid sequences listed in Table A; or a variant thereof.

The present invention also provides method of using anti-LEPR agonist antibodies and antigen-binding fragments thereof, comprising an HCVR and an LCVR amino acid sequence pair (HCVR/LCVR) comprising any of the HCVR amino acid sequences listed in Table A; or a variant thereof paired with any of the LCVR amino acid sequences listed in Table A; or a variant thereof. According to certain embodiments, the present invention provides anti-LEPR agonist antibodies and antigen-binding fragments thereof comprising an HCVR/LCVR amino acid sequence pair contained within any of the exemplary antibodies and fragments listed in Table A; or a variant thereof. In certain embodiments, the HCVR/LCVR amino acid sequence pair is selected from the group consisting of SEQ ID NOs: 2/10; 18/10; 26/10; 34/10; 42/10; 50/10; 58/10; 66/10; 74/10; 82/90; 98/90; and 106/90.

The present invention also provides method of using anti-LEPR agonist antibodies and antigen-binding fragments thereof, comprising a heavy chain CDR1 (HCDR1) comprising an amino acid sequence selected from any of the HCDR1 amino acid sequences listed in Table A; or a variant thereof.

The present invention also provides method of using anti-LEPR agonist antibodies and antigen-binding fragments thereof, comprising a heavy chain CDR2 (HCDR2) comprising an amino acid sequence selected from any of the HCDR2 amino acid sequences listed in Table A; or a variant thereof.

The present invention also provides method of using anti-LEPR agonist antibodies and antigen-binding fragments thereof, comprising a heavy chain CDR3 (HCDR3) comprising an amino acid sequence selected from any of the HCDR3 amino acid sequences listed in Table A; or a variant thereof.

The present invention also provides method of using anti-LEPR agonist antibodies and antigen-binding fragments thereof, comprising a light chain CDR1 (LCDR1) comprising an amino acid sequence selected from any of the LCDR1 amino acid sequences listed in Table A; or a variant thereof.

The present invention also provides method of using anti-LEPR agonist antibodies and antigen-binding fragments thereof, comprising a light chain CDR2 (LCDR2) comprising an amino acid sequence selected from any of the LCDR2 amino acid sequences listed in Table A; or a variant thereof.

The present invention also provides method of using anti-LEPR agonist antibodies and antigen-binding fragments thereof, comprising a light chain CDR3 (LCDR3) comprising an amino acid sequence selected from any of the LCDR3 amino acid sequences listed in Table A; or a variant thereof.

The present invention also provides method of using anti-LEPR agonist antibodies and antigen-binding fragments thereof, comprising an HCDR3 and an LCDR3 amino acid sequence pair (HCDR3/LCDR3) comprising any of the HCDR3 amino acid sequences listed in Table A; or a variant thereof, paired with any of the LCDR3 amino acid sequences listed in Table A; or a variant thereof.

The present invention also provides methods of using anti-LEPR agonist antibodies and antigen-binding fragments thereof, comprising a set of six CDRs (i.e., HCDR1, HCDR2 and HCDR3 of a HCVR and LCDR1, LCDR2, and LCDR3 of a LCVR) contained within any of the exemplary anti-LEPR antibodies listed in Table A; or a variant thereof. In certain embodiments, the HCDR1, HCDR2, HCDR3, LCDR1, LCDR2, and LCDR3 amino acid sequences set is selected from the group consisting of SEQ ID NOs: 4/6/8/12/14/16; 20/22/24/12/14/16; 28/30/32/12/14/16; 36/38/40/12/14/16; 44/46/48/12/14/16; 52/54/56/12/14/16; 60/62/64/12/14/16; 68/70/72/12/14/16; 76/78/80/12/14/16; 84/86/88/92/94/96; 100/102/104/92/94/96; and 108/110/112/92/94/96.

In a related embodiment, the present invention provides methods of using anti-LEPR agonist antibodies and antigen-binding fragments thereof, comprising a set of six CDRs (i.e., HCDR1, HCDR2, HCDR3, LCDR1, LCDR2, and LCDR3) contained within an HCVR/LCVR amino acid sequence pair as defined by any of the exemplary antibodies and fragments listed in Table A; or a variant thereof. For example, the present invention includes method of using anti-LEPR agonist antibodies and antigen-binding fragments thereof, comprising the HCDR1, HCDR2, HCDR3, LCDR1, LCDR2, and LCDR3 amino acid sequences set contained within an HCVR/LCVR amino acid sequence pair selected from the group consisting of SEQ ID NOs: 2/10; 18/10; 26/10; 34/10; 42/10; 50/10; 58/10; 66/10; 74/10; 82/90; 98/90; and 106/90. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition (discussed herein).

The present invention includes methods of using antibodies or antigen-binding fragments, that bind to the same epitope as an antibody or fragment specifically set forth herein (e.g., REGN4461, H4H16650P2, H4H16679P2, H4H17319P2, H4H17321P2, H4H18417P2, H4H18438P2, H4H18445P2, H4H18446P2, H4H18449P2, H4H18482P2, H4H18487P2 and/or H4H18492P2). See International Patent Application Publication No. WO2017/66204.

The present invention also includes methods of using antibodies and antigen-binding fragments that compete for binding to LEPR with an antibody or antigen-binding fragment that is specifically set forth herein (e.g., REGN4461, H4H16650P2, H4H16679P2, H4H17319P2, H4H17321P2, H4H18417P2, H4H18438P2, H4H18445P2, H4H18446P2, H4H18449P2, H4H18482P2, H4H18487P2 and/or H4H18492P2). The term “competes” as used herein, refers to an antibody or antigen-binding fragment that binds to an antigen (e.g., LEPR) and inhibits or blocks the binding of another antibody or antigen-binding fragment to the antigen. The term also includes competition between two antibodies or antigen-binding fragments, in both orientations, e.g., where a first antibody that binds and blocks binding of second antibody and vice versa. In certain embodiments, the first antibody or fragment and second antibody or fragment may bind to the same epitope. Alternatively, the first and second antibody or fragment may bind to different, but, for example, overlapping epitopes, wherein binding of one inhibits or blocks the binding of the second antibody or fragment, e.g., via steric hindrance. Competition between antibody or fragment may be measured by methods known in the art, for example, by a real-time, label-free bio-layer interferometry assay. Also, binding competition between anti-LEPR antibody or fragment can be determined using a real time, label-free bio-layer interferometry assay on an Octet RED384 biosensor (Pall ForteBio Corp.). See International Patent Application Publication No. WO2017/66204.

### Anti-LEPR Antibodies Comprising Fc Variants

According to certain embodiments of the present invention, methods for using anti-LEPR antibodies and antigen-binding fragments are provided wherein the antibody or fragment includes an Fc domain comprising one or more mutations which enhance or diminish antibody binding to the FcRn receptor, e.g., at acidic pH as compared to neutral pH. For example, the present invention includes anti-LEPR antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

For example, the present invention includes methods of using anti-LEPR antibodies and antigen-binding fragments comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); and 433K and 434F (e.g., H433K and N434F). All possible combinations of the foregoing Fc domain mutations, and other mutations within the antibody variable domains disclosed herein, are contemplated within the scope of the present invention.

The anti-LEPR antibodies and antigen-binding fragments that may be used in methods of the present invention may comprise a modified Fc domain having reduced effector function. As used herein, a “modified Fc domain having reduced effector function” means any Fc portion of an immunoglobulin that has been modified, mutated, truncated, etc., relative to a wild-type, naturally occurring Fc domain such that a molecule comprising the modified Fc exhibits a reduction in the severity or extent of at least one effect selected from the group consisting of cell killing (e.g., ADCC and/or CDC), complement activation, phagocytosis and opsonization, relative to a comparator molecule comprising the wild-type, naturally occurring version of the Fc portion. In certain embodiments, a “modified Fc domain having reduced effector function” is an Fc domain with reduced or attenuated binding to an Fc receptor (e.g., FcγR).

In certain embodiments of the present invention, the modified Fc domain is a variant IgG1 Fc or a variant IgG4 Fc comprising a substitution in the hinge region. For example, a modified Fc for use in the context of the present invention may comprise a variant IgG1 Fc wherein at least one amino acid of the IgG1 Fc hinge region is replaced with the corresponding amino acid from the IgG2 Fc hinge region. Alternatively, a modified Fc for use in the context of the present invention may comprise a variant IgG4 Fc wherein at least one amino acid of the IgG4 Fc hinge region is replaced with the corresponding amino acid from the IgG2 Fc hinge region. Non-limiting, exemplary modified Fc regions that can be used in the context of the present invention are set forth in US Patent Application Publication No. 2014/0243504.

Other modified Fc domains and Fc modifications that can be used in the context of the present invention include any of the modifications as set forth in US 2014/0171623; U.S. Pat. No. 8,697,396; US 2014/0134162; WO 2014/043361. Methods of constructing antibodies or other antigen-binding fusion proteins comprising a modified Fc domain as described herein are known in the art.

### Preparation of Human Antibodies

The anti-LEPR antibodies and antigen-binding fragments that may be used in methods of the present invention can be fully human antibodies. Methods for generating monoclonal antibodies, including fully human monoclonal antibodies are known in the art. Any such known methods can be used in the context of the present invention to make human antibodies that specifically bind to human LEPR.

Using VELOCIMMUNE™ technology, for example, or any other similar known method for generating fully human monoclonal antibodies, high affinity chimeric antibodies to LEPR are initially isolated having a human variable region and a mouse constant region. As in the experimental section below, the antibodies are characterized and selected for desirable characteristics, including affinity, ligand blocking activity, selectivity, epitope, etc. If necessary, mouse constant regions are replaced with a desired human constant region, for example wild-type or modified IgG1 or IgG4, to generate a fully human anti-LEPR antibody. While the constant region selected may vary according to specific use, high affinity antigen-binding and target specificity characteristics reside in the variable region. In certain instances, fully human anti-LEPR antibodies are isolated directly from antigen-positive B cells.

See International patent application publication no. WO2017/066204.

### Combinations and Pharmaceutical Compositions

The present invention provides methods for using compositions that include anti-LEPR antibodies and antigen-binding fragments and one or more ingredients.

To prepare pharmaceutical compositions of the anti-LEPR antibodies and antigen-binding fragments thereof (e.g., REGN4461, H4H16650P2, H4H16679P2, H4H17319P2, H4H17321P2, H4H18417P2, H4H18438P2, H4H18445P2, H4H18446P2, H4H18449P2, H4H18482P2, H4H18487P2 and/or H4H18492P2), the antibodies or fragments are admixed with a pharmaceutically acceptable carrier or excipient. See, e.g., Remington's Pharmaceutical Sciences and U.S. Pharmacopeia: National Formulary, Mack Publishing Company, Easton, Pa. (1984); Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y. In an embodiment of the invention, the pharmaceutical composition is sterile. The use of such compositions is part of the present invention.

Pharmaceutical compositions for use in methods of the present invention include pharmaceutically acceptable carriers, diluents, excipients and/or stabilizers, such as, for example, water, buffering agents, stabilizing agents, preservatives, isotonifiers, non-ionic detergents, antioxidants and/or other miscellaneous additives.

The scope of the present invention includes methods for using desiccated, e.g., freeze-dried, compositions comprising an anti-LEPR antigen-binding protein, e.g., antibody or antigen-binding fragment thereof (e.g., REGN4461, H4H16650P2, H4H16679P2, H4H17319P2, H4H17321P2, H4H18417P2, H4H18438P2, H4H18445P2, H4H18446P2, H4H18449P2, H4H18482P2, H4H18487P2 and/or H4H18492P2), or a pharmaceutical composition thereof that includes a pharmaceutically acceptable carrier but substantially lacks water. For example, such desiccated compositions can be reconstituted, e.g., with water, and then administered to a patient.

In a further embodiment of the invention, a further therapeutic agent is administered to a patient in association with an anti-LEPR antibody or antigen-binding fragment thereof (e.g., REGN4461, H4H16650P2, H4H16679P2, H4H17319P2, H4H17321P2, H4H18417P2, H4H18438P2, H4H18445P2, H4H18446P2, H4H18449P2, H4H18482P2, H4H18487P2 and/or H4H18492P2), disclosed herein is administered to the patient in accordance with the Physicians' Desk Reference 2003 (Thomson Healthcare; 57^{th} edition (Nov. 1, 2002)).

The mode of administration of an antibody or antigen-binding fragment thereof or composition thereof can vary. Routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal or intra-arterial. Methods of the present invention include uses wherein a LEPR agonist is administered to a patient by any such route.

The present invention provides methods of use including the step of administering an anti-LEPR agonist antibody or antigen-binding fragment thereof (e.g., REGN4461, H4H16650P2, H4H16679P2, H4H17319P2, H4H17321P2, H4H18417P2, H4H18438P2, H4H18445P2, H4H18446P2, H4H18449P2, H4H18482P2, H4H18487P2 and/or H4H18492P2) to a patient, comprising introducing the antibody or fragment or a pharmaceutical composition or combination thereof into the body of the patient. For example, in an embodiment of the invention, the method comprises piercing the body of the patient, e.g., with a needle of a syringe, and injecting the antigen-binding protein or a pharmaceutical composition or combination thereof into the body of the patient, e.g., into the vein, artery, tumor, muscular tissue or subcutis of the patient.

The present invention includes methods of using combinations including an anti-LEPR agonist antibodies or antigen-binding fragments thereof (e.g., REGN4461, H4H16650P2, H4H16679P2, H4H17319P2, H4H17321P2, H4H18417P2, H4H18438P2, H4H18445P2, H4H18446P2, H4H18449P2, H4H18482P2, H4H18487P2 and/or H4H18492P2), in association with one or more further therapeutic agents. The LEPR agonist (e.g., anti-LEPR agonist antibodies and antigen-binding fragments) and the further therapeutic agents can be in a single composition or in separate compositions. For example, in an embodiment of the invention, the further therapeutic agent is human leptin, metreleptin, a PCSK9 inhibitor (e.g., an anti-PCSK9 antibody, for example, alirocumab, evolocumab, bococizumab, lodelcizumab or ralpancizumab), an HMG-CoA reductase inhibitor (e.g., atorvastatin, rosuvastatin, cerivastatin, pitavastatin, fluvastatin, simvastatin, lovastatin or pravastatin), ezetimibe, insulin, an insulin variant, an insulin secretagogue, metformin, a sulfonylurea, a sodium glucose cotransporter 2 (SGLT2) inhibitor (e.g., dapaglifozin, canaglifozin or empagliflozin), a selective agonist of the MC₄ receptor (e.g., setmelanotide), a GLP-1 agonist or analogue (e.g., extendin-4, exenatide, liraglutide, lixisenatide, albiglutide or dulaglutide), a glucagon (GCG) inhibitor (e.g., an anti-GCG antibody), a glucagon receptor (GCGR) inhibitor (e.g., an anti-GCGR antibody, a small molecule GCGR antagonist, a GCGR-specific antisense oligonucleotide, an anti-GCGR aptamer, for example, a spiegelmer, an angiopoietin-like protein (ANGPTL) inhibitor (e.g., an anti-ANGPTL3 antibody, an anti-ANGPTL4 antibody, an anti-ANGPTL8 antibody), phentermine, orlistat, topiramate, bupropion, topiramate and phentermine, bupropion and naltrexone, bupropion and zonisamide, pramlintide and metrelepin, lorcaserin, cetilistat, tesofensine or velneperit. In an embodiment of the invention, the further therapeutic agent is a pain reliever, e.g., a non-steroidal anti-inflammatory drug (NSAID), aspirin, acetaminophen, ibuprofen, naproxen, a corticosteroid, a muscle relaxant, a COX2 inhibitor, an analgesic, a non-opioid, an anti-depressant, an anxiolytic, acetaminophen, an opioid or diclofinac.

The term “in association with” indicates that components, an anti-LEPR agonist antibody or antigen-binding fragment thereof of the present invention, along with another agent such as metreleptin, can be formulated into a single composition, e.g., for simultaneous delivery, or formulated separately into two or more compositions (e.g., a kit including each component for simultaneous delivery). Each component can be administered to a patient at a different time than when the other component is administered; for example, each administration may be given non-simultaneously (e.g., separately or sequentially) at intervals over a given period of time. Moreover, the separate components may be administered to a patient by the same or by a different route.

### Treatment and Administration

The LEPR agonists (e.g., anti-LEPR agonist antibodies and antigen-binding fragments set forth herein) can be used to reduce pain and/or the use of analgesics such as opioids in a patient rapidly, for example, which is associated with a leptin deficiency or leptin resistance condition, e.g., lipodystrophy or obesity. The reduction of such pain can lead to improvements in quality of life as well as reduction in the use of analgesics including opioids.

The present invention includes methods for treating or preventing pain in a patient, e.g., who is suffering from a leptin deficiency or leptin resistance condition, e.g., a lipodystrophy or obesity, comprising administering an effective amount of LEPR agonist, such as anti-LEPR agonist antibody or antigen-binding fragment thereof (e.g., REGN4461, H4H16650P2, H4H16679P2, H4H17319P2, H4H17321P2, H4H18417P2, H4H18438P2, H4H18445P2, H4H18446P2, H4H18449P2, H4H18482P2, H4H18487P2 and/or H4H18492P2), to the patient.

An effective amount or dose of LEPR agonist administered to a patient may vary depending upon the age and the size of the patient, target disease, conditions, route of administration, and the like. The preferred dose can be calculated according to body weight or body surface area. In an adult patient, it may be advantageous to intravenously administer the LEPR agonist (e.g., anti-LEPR agonist antibody or antigen-binding fragment) at a single dose of about 0.01 to about 20 mg/kg body weight Depending on the severity of the condition, the frequency and the duration of the treatment can be adjusted. Effective dosages and schedules for administering LEPR agonist may be determined empirically; for example, patient progress can be monitored by periodic assessment, and the dose adjusted accordingly. Moreover, interspecies scaling of dosages can be performed using well-known methods in the art (e.g., Mordenti et al., Interspecies Scaling of Clearance and Volume of Distribution Data for Five Therapeutic Proteins, Pharmaceut. Res. 8:1351-1359 (1991).

In an embodiment of the invention, the pain, anxiety and/or depression reduction observed occurs, in the patient, within less than 1, 1, 2, 3, 4 or 5 days (or less) of the first effective dose of LEPR agonist (e.g., REGN4461, H4H16650P2, H4H16679P2, H4H17319P2, H4H17321P2, H4H18417P2, H4H18438P2, H4H18445P2, H4H18446P2, H4H18449P2, H4H18482P2, H4H18487P2 and/or H4H18492P2), for example, of about 5 mg/ml intravenous and 300 mg subcutaneously thereafter.

The present invention also provides methods for reducing or maintaining a reduction in the use of analgesics (e.g., opioids, e.g., oxycodone or gabapentin), the use of anxiolytics, the use of anti-depressants, analgesia seeking behavior, pro re nata use of analgesia, analgesia overdose, and/or death due to abuse of analgesia in a patient, e.g., who is suffering from a leptin deficiency or leptin resistance condition, by administering an effective amount of LEPR agonist such as REGN4461. For example, in an embodiment of the invention, a patient ceases or the treating physician orders the cessation of the regular use of analgesics (e.g., opioids, e.g., oxycodone or gabapentin) very close in time to the initiation of treatment with the LEPR agonist; for example, simultaneously or within 1-2 days. In another embodiment of the invention, the patient reduces the usage of (e.g., reduces the dosage amount and/or frequency of dosage) or the treating physician orders the reduction of usage of (e.g., reduces the dosage amount and/or frequency of dosage) analgesics (e.g., opioids, e.g., oxycodone or gabapentin) very close in time to the initiation of treatment with the LEPR agonist; for example, simultaneously or within 1-2 days. In such a case, wherein the use of analgesics (e.g., opioids, e.g., oxycodone or gabapentin) is reduced or ceased or ordered to be reduced or ceased, such reduction or cessation is maintained (e.g., for 6 months or a year or 1½ years or two years or more) but for the episodic use (e.g., for 1, 2 or 3 days or less; or for 1-3 days) of analgesics when needed to treat a particularly painful medical issue (e.g., abdominal, liver or pancreatic pain). Where analgesic (e.g., opioids, e.g., oxycodone or gabapentin) use is reduced (or ordered as such by the physician), further reductions (e.g., eventually leading to a cessation) may occur or be ordered over time. For example, in an embodiment of the invention, the analgesics are opioids such as oxycodone, but the patient is permitted to continue to use non-opioid analgesics (e.g., paracetamol) as needed.

The present invention further provides methods for reducing hospitalization, emergency room use or the need for emergency medical treatment or doctor visits or the need for medical attention of/by a patient, e.g., that suffers from a leptin deficiency or leptin resistance condition (e.g., a lipodystrophy such as PLD), due to pain (e.g., due to pancreatitis), abdominal pain, pancreatitis, anxiety and/or depression (e.g., which is associated with the condition) by administering an effective amount of LEPR agonist (e.g., REGN4461).

The present invention also provides methods for treating or preventing centralized pain or central pain syndrome (e.g., chronic centralized pain). Central pain syndrome is a neurological condition caused by damage to or dysfunction of the central nervous system (CNS), which includes the brain, brainstem, and spinal cord. This syndrome can be caused by stroke, multiple sclerosis, tumors, epilepsy, brain or spinal cord trauma, or Parkinson's disease. The character of the pain associated with this syndrome differs widely among individuals partly because of the variety of potential causes. Central pain syndrome may affect a large portion of the body or may be more restricted to specific areas, such as hands or feet. The extent of pain may be usually related to the cause of the CNS injury or damage. Pain can be constant, may be moderate to severe in intensity, and is often made worse by touch, movement, emotions, and temperature changes, e.g., cold temperatures. Individuals experience one or more types of pain sensations, the most prominent being burning. Mingled with the burning may be sensations of “pins and needles;” pressing, lacerating, or aching pain; and brief, intolerable bursts of sharp pain similar to the pain caused by a dental probe on an exposed nerve. Individuals may have numbness in the areas affected by the pain. The burning and loss of touch sensations are usually most severe on the distant parts of the body, such as the feet or hands. Central pain syndrome often begins shortly after the causative injury or damage, but may be delayed by months or even years, especially if it is related to post-stroke pain. Thus, the present invention includes methods for treating or preventing central pain, chronic central pain, forms of pain involving centralization and/or hypersensitization of pain by the central nervous system or central pain syndrome in a patient (e.g., in a patient suffering from a leptin deficiency or leptin resistance condition, e.g., a lipodystrophy or obesity), comprising administering an effective amount of anti-LEPR agonist antibody or antigen-binding fragment thereof (e.g., REGN4461, H4H16650P2, H4H16679P2, H4H17319P2, H4H17321P2, H4H18417P2, H4H18438P2, H4H18445P2, H4H18446P2, H4H18449P2, H4H18482P2, H4H18487P2 and/or H4H18492P2) to the patient.

In an embodiment of the invention, an effective amount of LEPR agonist (e.g., REGN4461, H4H16650P2, H4H16679P2, H4H17319P2, H4H17321P2, H4H18417P2, H4H18438P2, H4H18445P2, H4H18446P2, H4H18449P2, H4H18482P2, H4H18487P2 and/or H4H18492P2) is such that a blood concentration of the agonist is about 100 mg/liter (or more).

In an embodiment of the invention, an effective amount of LEPR agonist (e.g., REGN4461, H4H16650P2, H4H16679P2, H4H17319P2, H4H17321P2, H4H18417P2, H4H18438P2, H4H18445P2, H4H18446P2, H4H18449P2, H4H18482P2, H4H18487P2 and/or H4H18492P2) is one or more intravenous (IV) doses of about 5 mg/kg and one or more subcutaneous (SC) doses of about 250-300 mg once weekly thereafter (e.g., wherein one IV dose is given on day 1 and the SC dosing initiates on day 5 and continues weekly thereafter).

A leptin deficiency or leptin resistance condition includes, for example, monogenic obesity, obesity, impaired thyroid function, early onset obesity, dyslipidemia, hypogonadism, reproductive dysfunction, hyperphagia and impaired satiety, impaired immune function (e.g., CD4⁺ counts), metabolic dysfunction, no or irregular menses, metabolic syndrome, diet-induced food craving, functional hypothalamic amenorrhea, type 1 diabetes, type 2 diabetes, insulin resistance, severe insulin resistance including severe insulin resistance due to mutation in insulin receptor, severe insulin resistance not caused by mutation in the insulin receptor, severe insulin resistance caused by a mutation in downstream signaling pathways or induced by other causes, non-alcoholic and alcoholic fatty liver diseases, Alzheimer's disease, leptin deficiency, leptin resistance, lipodystrophies, Leprechaunism/Donohue syndrome and Rabson-Mendenhall syndrome.

Another leptin deficiency or leptin resistance condition includes the possession of neutralizing anti-leptin autoantibodies.

Another leptin deficiency or leptin resistance condition includes hypoleptinemia, female infertility, amenorrhea, abnormal hormone cycle, impaired immune function, or hypothyroidism.

Lipodystrophies include, for example, partial lipodystrophy (PLD), congenital generalized lipodystrophy, acquired generalized lipodystrophy, familial partial lipodystrophy, acquired partial lipodystrophy, centrifugal abdominal lipodystrophy, lipoatrophia annularis, localized lipodystrophy and HIV-associated lipodystrophy.

In an embodiment of the invention, a patient suffering from a leptin deficiency or leptin resistance condition has a genotype characterized by a LEPR mutant that exhibits no signaling in the presence of leptin (a signaling-defective LEPR mutant). An exemplary signaling-defective LEPR mutation is LEPR-A409X, e.g., LEPR-A409E (Farooqi et al., Clinical and Molecular Genetic Spectrum of Congenital Deficiency of the Leptin Receptor, N Engl J Med 356(3): 237-247 (2007)). In an embodiment of the invention, a patient suffering from a leptin deficiency or leptin resistance condition has a genotype characterized by a LEPR mutant that exhibits reduced signaling in the presence of leptin (as compared to wild-type LEPR) which may be referred to as a “signaling-impaired LEPR mutant.” An exemplary signaling-impaired LEPR mutation is LEPR-P316X, e.g., LEPR-P316T (Mazen et al., Homozygosity for a novel missense mutation in the leptin receptor gene (P316T) in two Egyptian cousins with severe early onset obesity, Mol Genet Metab 102 (4):461-464 (2011)). In an embodiment of the invention, the LEPR mutation is LEPR-L372X, e.g., LEPR-L372A.

In an embodiment of the invention, the pain suffered by a patient with a leptin deficiency or leptin resistance condition such as a lipodystrophy (e.g. PLD) is acute pain, chronic pain, abdominal pain, liver pain, painful subcutaneous nodules or maculopapular lesions associated with acquired generalized lipodystrophy of the Panniculitis variety (type 1), localized pain e.g., in the back, shoulder, arm and/or neck, associated with the accumulation of excess fat deposits at spots on the body of a patient suffering from a lipodystrophy. For example, a painful “buffalo hump,” or dorsocervical fat pad on the neck can be a source of pain (e.g., in the neck as well as in the back and shoulder) in some suffering from lipodystrophy. In an embodiment of the invention, the source of pain is neuropathy, arthritis, chronic back pain, fibromyalgia or myopathy. Thus, the present invention includes methods for treating or preventing any such pain, for example, myopathy pain, (e.g., in a patient suffering from a leptin deficiency or leptin resistance condition) as set forth herein.

In an embodiment of the invention, the abdominal pain is right hypochondrial pain.

In an embodiment of the invention, the patient with abdominal and/or back pain (e.g., abdominal pain radiating to the back or vice versa) suffers from oedematous pancreas, necrotic pancreatic tissue, Ileitis, gastroenteritis, renal pain (left and/or right), pancreatitis (e.g., acute pancreatitis), biliary pain, eosinophilia, chylomicronemia, acute cholecystitis, liver inflammation, cirrhosis, liver failure, dyspepsia, liver pain, hepatitis, nonalcoholic fatty liver disease (NAFLD), and/or a hepatic steatosis.

In an embodiment of the invention, a patient receiving a LEPR agonist in accordance with the present invention, e.g., for the reduction of pain, also demonstrates:
a reduction in hepatic steatosis (e.g., as estimated by magnetic resonance imaging proton density fat fraction (PDFF));
a reduction in liver enzyme levels in the blood;
a reduction in liver size (e.g., as estimated by physical examination)—for example, the longitudinal diameter as measured craniocaudally from the uppermost right hemi-diaphragm to the inferior tip of the right lobe;
a reduction in hepatic stiffness (e.g., as measured by hepatic elastography); and/or
a reduction in spleen size.

Methods for achieving any such reduction, in a patient (e.g., suffering from a leptin deficiency or leptin resistance condition), by administering an effective amount of LEPR agonist to the patient is part of the present invention.

A “patient” of “subject” is a mammal (e.g., a monkey, non-human primate, mouse, rat or rabbit), preferably a human. In an embodiment of the invention, the patient suffers from a lipodystrophy, partial lipodystrophy and/or obesity. In an embodiment of the invention, the patient or subject has any one or more of the following characteristics or a history thereof:
A positive anti-GAD65 titer;
Abnormal growth hormone secretion;
About 3.2 ng/ml leptin blood levels;
Adiposity;
Adrenarche;
Alanine aminotransferase (ALT) serum levels of about 80-120 IU/liter;
Aspartate Aminotransferase (AST) serum levels of about 80-160 IU/liter;
Blood glucose of about 200-500 (200, 300, 400 or 500) mg/dl;
Delay in growth;
Delay in pubertal growth spurt;
Dyslipidemia;
Elevated HbA1c;
Estradiol levels of less than about 20 pg/ml (e.g., in a female patient);
Follicle stimulating hormone (FSH) levels of about 1-2 mU/ml (e.g., in a female patient);
HbA1c percentage of about 9-10;
Hepatic fat content (proton-density fat-fraction (PDFF)) of about 30% (e.g., as measured by the Dixon method (Dixon, Simple proton spectroscopic imaging.

Radiology 1984; 153:189-194);
Hepatomegaly;
Hypercholesterolemia;
Hyperglycemia;
Hyperinsulinemia;
Hyperphagia;
Hypertriglyceridemia (e.g., blood levels of about 1200 mg/dl);
Insulin resistance;
Insulin use, e.g., at a daily dose of about 400-1600 (400, 500, 600, 700, 800, 900, 1000, 1100, 1200, 1300, 1400, 1500 or 1600) units;
Lack of breast development;
Low bone mineral content;
Low bone mineral density (or low bone mass);
Low lean body mass;
Luteinizing hormone (LH) levels of about 0.1 pg/ml (e.g., in a female patient);
Neutralizing antibodies against metreleptin;
Nonalcoholic steatohepatitis (NASH);
Obesity;
Pancreatitis;
Plasmapheresis;
Possession of anti-leptin or anti-metreleptin neutralizing antibodies;
Testosterone levels of less than about 0.05 ng/ml (e.g., in a female patient);
Use of gabapentin, oxycodone, venlafaxine, buspirone, paracetamol, ketamin, ketorolac, lorazepam and/or morphine; and/or
Use of metreleptin and/or setmelanotide or a discontinuation of such use;

### Analgesics

Analgesics are agents used for the reduction of pain. Patients suffering from a leptin deficiency or leptin resistance condition, such as a lipodystrophy, can discontinue or reduce the use of analgesics, e.g., opioids and non-opioids, such as CGRP inhibitors, COX-2 inhibitors, salicylates, acetaminophen (paracetamol) and NSAIDs, when treated with a LEPR agonist such as REGN4461, H4H16650P2, H4H16679P2, H4H17319P2, H4H17321P2, H4H18417P2, H4H18438P2, H4H18445P2, H4H18446P2, H4H18449P2, H4H18482P2, H4H18487P2 and/or H4H18492P2.

In an embodiment of the invention, pain suffered by a patient with a leptin deficiency or leptin resistance condition is “chronic” and has been treated with chronic use of analgesics such as opioids. Chronic pain or chronic use of a therapy, such as analgesics, refers to recurring or on-going instances of pain or therapy (e.g., analgesic) use for a long period of time (e.g., a year or more) and with no definitive end-point.

Episodic pain or episodic use of a therapy, such as analgesics, refers to pain occurring or therapeutic (e.g., analgesic) use occurring in a single, discrete instance of one to several days in duration and with a definitive end-point intended. A patient can suffer multiple episodes of pain and engage in multiple episodes of therapeutic (e.g., analgesic) use over a given period of time.

In an embodiment of the invention, the pain is localized pain, localized abdominal pain, generalized pain or generalized abdominal pain. Generalized abdominal pain means that a patient feels pain in more than half of the abdomen.

Opioid seeking behavior and the use of opioids is associated with pain caused by, for example, a lipodystrophy condition. Use of opioids can lead to overuse, overdose and death. The reduction of pain in patients suffering from a leptin deficiency or leptin resistance condition can lead to a reduction of such opioid seeking behavior and use, thus leading to fewer overdoses and death. Thus, the present invention includes methods for reducing opioid seeking behavior and/or the use of opioids, overuse of opioids, overdosage of opioids, addiction to opioids and/or deaths caused by opioid use comprising treating or preventing pain in a patient (e.g., suffering from a lipodystrophy or obesity) comprising administering an effective amount of LEPR agonist (e.g., REGN4461, H4H16650P2, H4H16679P2, H4H17319P2, H4H17321P2, H4H18417P2, H4H18438P2, H4H18445P2, H4H18446P2, H4H18449P2, H4H18482P2, H4H18487P2 and/or H4H18492P2) to the patient.

The chemical structure of opioids is subdivided into those based on (i) the 4,5-epoxymorphinan ring, such as morphine

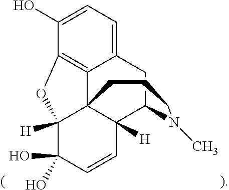

codeine, oxymorphone, oxycodone, buprenorphine, hydromorphone and hydrocodone, (ii) the phenylpiperidines such as alfentanil, fentanyl

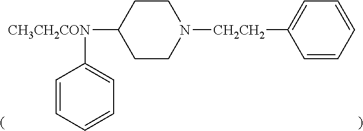

and sufentanil and (iii) the diphenylheptylamines such as methadone

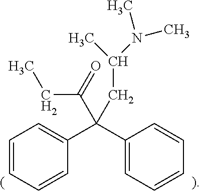 [SMILES: CCC(=O)C(CC(C)N(C)C)(c1ccccc1)c1ccccc1]

See e.g., Drewes, Br. J. Clin. Pharmacol. 75(1): 60-78 (2012). Although these compounds differ in chemical structure, physicochemical properties and in pharmacokinetics they have one common feature, which is their interaction with the mu (μ) opioid receptor as the primary target. In an embodiment of the invention, the opioid is alfentanil, codeine, concentrate of poppy straw, dextromoramide, dextropropoxyphene, dihydrocodeine, dihydroetorphine, diphenoxylate, ethylmorphine, etorphine, fentanyl, heroin, hydrocodone, hydromorphone, ketobemidone, levorphanol, methadone, methadone, morphine, morphine-n-oxide, nicomorphine, norcodeine, opium, oripavine, oxycodone, oxymorphone, pethidine, pethidine intermediate, pholcodine, piritramide, remifentanil, sufentanil, thebaine or tilidine.

Calcitonin gene-related peptide (CGRP) inhibitors block the effect of CGRP, which is a small protein that is highly prevalent in the sensory nerves that supply the head and the neck. CGRP is involved in pain transmission and levels increase during a migraine attack. It may also play a causative role in the induction of migraine attacks. CGRP inhibitors are used for the management of migraine. Two types of CGRP inhibitors include monoclonal antibodies (e.g., eptinezumab, galcanezumab, erenumab and fremanezumab) and CGRP receptor antagonists (gepants). Gepants are small molecule drugs which block the CGRP receptor and are effective at both relieving migraines and preventing them. Gepants include ubrogepant and rimegepant sulfate.

Cyclooxygenase-2 (COX-2) inhibitors are a type of nonsteroidal anti-inflammatory drug (NSAID) that specifically blocks COX-2 enzymes. COX-2 inhibitors include valdecoxib and celecoxib.

A salicylate is a salt or ester of salicylic acid. Salicylates are found naturally in some plants (such as white willow bark and wintergreen leaves) and are thought to protect the plant against insect damage and disease. Aspirin is a derivative of salicylic acid—and is also known as acetylsalicylic acid. Salicylates include magnesium salicylate, aspirin, choline salicylate & magnesium salicylate, diflunisal, salsalate and aspirin & citric acid & sodium bicarbonate.

Nonsteroidal anti-inflammatory agents (usually abbreviated to NSAIDs) are a group of medicines that relieve pain and fever and reduce inflammation. NSAIDs include meclofenamat, ketoprofen, fenoprofen, tolmetin, diclofenac & misoprostol, piroxicam, indomethacin, diclofenac, etodolac, ibuprofen, flurbiprofen, sulindac, ketorolac, naproxen, diflunisal, famotidine & ibuprofen, meloxicam, oxaprozin, esomeprazole & naproxen, nabumetone, mefenamic acid, bromfenac and phenylbutazone. Other analgesics include acetaminophen.

### Anti-Depressants

Patients suffering from a leptin deficiency or leptin resistance condition such as lipodystrophy can discontinue or reduce the use of anti-depressants, e.g., SSRIs, SNRIs, atypical antidepressants, TCAs and MAOIs, when treated with a LEPR agonist such as REGN4461, H4H16650P2, H4H16679P2, H4H17319P2, H4H17321P2, H4H18417P2, H4H18438P2, H4H18445P2, H4H18446P2, H4H18449P2, H4H18482P2, H4H18487P2 and/or H4H18492P2.

Selective serotonin reuptake inhibitors (SSRIs) inhibit the reabsorption of serotonin back into the nerve cells (called reuptake). This mechanism results in higher levels of active serotonin in the brain. SSRIs include fluoxetine, paroxetine, sertraline, citalopram and escitalopram

Serotonin and norepinephrine reuptake inhibitors (SNRIs) are a class of drugs used primarily to treat depression, though certain SNRIs are also indicated for treatment of anxiety and chronic pain associated with diabetic neuropathy and fibromyalgia. SNRIs work by blocking the reabsorption (or reuptake) of serotonin and norepinephrine back into the nerve cells that released them, which increases the levels of active neurotransmitters in the brain. Examples of SNRI medications include duloxetine, venlafaxine, desvenlafaxine and levomilnacipran.

Atypical antidepressants do not fit neatly into any of the other antidepressant categories. These antidepressants include, for example, trazodone, mirtazapine, vortioxetine, vilazodone and bupropion.

Tricyclic antidepressants (TCAs) are characterized by a core three-ring chemical structure. Typically, individual TCAs differ in their substitution of carbon or nitrogen in the central ring, and in the radicle on the amine chain. TCAs include imipramine, nortriptyline, amitriptyline, doxepin and desipramine.

Monoamine oxidase inhibitors (MAOIs) ease depression by affecting chemical messengers (neurotransmitters) used to communicate between brain cells. An enzyme called monoamine oxidase is involved in removing the neurotransmitters norepinephrine, serotonin and dopamine from the brain. MAOIs prevent this from happening, which makes more of these brain chemicals available to effect changes in both cells and circuits that have been impacted by depression. MAOIs include selegiline, tranylcypromine, phenelzine and isocarboxazid.

### Anxiolytics

Anxiolytics are medicines that work on the central nervous system to relieve anxiety, aid sleep, or have a calming effect. Some benzodiazepines and their derivatives can be used to treat anxiety. Benzodiazepines include alprazolam, clonazepam, diazepam, and lorazepam, estazolam, flurazepam, quazepam, temazepam, triazolam and alprazolam. All benzodiazepines are thought to work by enhancing the inhibitory action of γ-aminobutyric acid (GABA). Other drug classes that are also considered effective at relieving anxiety include the SSRIs, SNRIs, tricyclic antidepressants and buspirone. Other drug classes that have a sedative effective which may be used to treat anxiety include first-generation antihistamines, agonists of melatonin receptors, anesthetics, eszopiclone, zaleplon, zolpidem, zopiclone, and several others.

### EXAMPLES

The following examples are put forth so as to provide those of ordinary skill in the art with a complete disclosure and description of how to make and use the methods and compositions of the invention, and are not intended to limit the scope of what the inventors regard as their invention.

### Example 1: LEPR Agonist mAb Treatment is Associated with Pain Relief, Reduced Body Weight, Serum Triglycerides and Hepatic Steatosis in a Patient with Partial Lipodystrophy

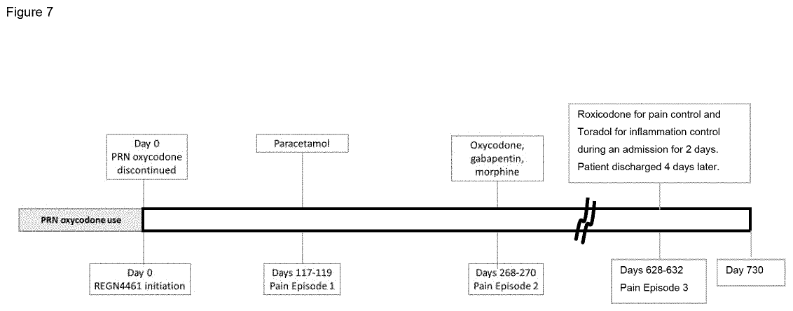

This example summarizes the case of a patient suffering from partial lipodystrophy (PLD). The patient was a regular pro re nata (PRN) user of oxycodone which was discontinued upon the initiation of REGN4461 treatment. PRN oxycodone use (prior to the initiation of REGN4461) was at least 2 to 3 doses for at least 3 days of the week for at least 12 months. But for periodic pain episodes, regular oxycodone usage was successfully ceased. Moreover, two days prior to the REGN4461 treatment, the dosage of gabapentin treatment for pain was reduced. The patient managed to reduce the gabapentin dosage 6 days later and again on day 7 and to maintain this reduction. A brief summary of the patient's opioid use is set forth in FIG. 7.

Compassionate treatment with the LEPR agonist, REGN4461, was sought for a patient with atypical partial lipodystrophy and a complicated disease course. Briefly, this patient had partial lipodystrophy with atypical presentation. The atypical features included the presence of type 1 diabetes, early-onset severe steatohepatitis with stage 4 fibrosis, and later development of additional autoimmune features. At age 13, she received metreleptin for 12 months as part of a clinical research protocol (NCT01679197). No serious adverse events were noted at that time. She continued metreleptin in an extension protocol (NCT02654977) due to perceived clinical benefit as reported previously. At around 18 months of therapy, her clinical status acutely deteriorated and further testing confirmed the presence of neutralizing antibodies against metreleptin. The inability to correct metabolic complications and the patient's serious disease necessitated a search for an alternative treatment strategy that had the potential to exert its effect independent of leptin action. The first attempt in this pursuit, with setmelanotide (first in class MC4R agonist), did not result in any metabolic benefit.

Because the patient's status continued to deteriorate, and she was recurrently hospitalized due to pancreatitis and pain, in part due to underlying severe refractory hypertriglyceridemia and massive hepatomegaly, compassionate treatment with the LEPR agonist mAb, REGN4461, was sought under an investigator-initiated IND.

REGN4461 comprises the heavy and light chain immunoglobulins set forth below:

REGN4461 heavy chain
(SEQ ID NO: 113)
QVQLVESGGSVVQPGRSLRLSCAASGFTFSTYAMYWVRQTPGKGLEWVAV
LYSDGSNKYYIDSVKGRFTISRDTSTNTLYLQMSSLRADDSALYYCARLN
WDYWYFDLWGRGTLVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK
REGN4461 light chain
(SEQ ID NO: 114)
DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC

The patient's initial blood chemistry values are set forth below in Table 1-1.

TABLE 1-1
Lab Tests at Initial Evaluation Period

| Parameter | Value | Reference values |
|---|---|---|
| HbA1c (%, mmol/mol) | 5.8 (40) | 4.2-5.8 (22-40) |
| Glucose (mg/dL) | 99 | 70-100 |
| Insulin (uU/mL) | 225.4 | 1.0-21.0 |
| Triglyceride (mg/dL) | 305 | <130 |
| Total cholesterol (mg/dL) | 169 | <170 |
| HDL cholesterol (mg/dL) | 29 | <40 |
| LDL cholesterol (mg/dL) | 79 | <90 |
| Chol/HDL Ratio | 5.8 | <4.5 |
| Apo-lipoprotein A-1 (mg/dL) | 101 | >110 |
| Apo-lipoprotein B (mg/dL) | 109 | <105 |
| Creatinine (mg/dL) | 0.44 | 0.4-0.9 |
| AST (IU/L) | 53 | 5-60 |
| ALT(IU/L) | 73 | <35 |
| ALK (IU/L) | 348 | 90-420 |
| GGTP (IU/L) | 41 | 8-35 |
| CK (IU/L) | 113 | 26-180 |
| Leptin (ng/mL) | 1.2 | NA |
| IGF-1 (ng/mL) | 220 | 143-693 |
| Cortisol (ug/dL) | 9.4 | A.M.: 7.0-22.0 |
| FSH (mIU/mL) | 3.7 | 2.0-12.0 |
| LH (mIU/mL) | 0.4 | 2-15 |
| Estradiol (pg/mL) | 13 | Prepubertal: <20 |
| Testosterone (ng/mL) | 0.06 | 0.10-0.70 |
| C3 Complement (mg/dL) | 111 | 79-152 |
| C4 Complement (mg/dL) | 15.1 | 16-38 |
| Antinuclear Antibody | 1/320 (speckled) | Negative |
| GAD Antibody (units/mL) | >250 | 0-5 |

The patient received her first intravenous infusion of REGN4461 on day 1 of week 1 and continued subcutaneous injection on a weekly basis starting on day 5 of week 1. The treatment consisted of 5 mg/kg intravenous infusion followed by 300 mg weekly subcutaneous injections of the LEPR agonist, REGN4461. The SC dosage given in this regimen was increased to 450 mg at week 82 and an additional IV loading dose was given at week 83. A detailed description of outcome measures, assessments and detailed schedule of events can be found Table 1-2.

TABLE 1-2
Schedule of Assessments for Treatment Period 1 (A), Period 2 (B) and the
Extended Treatment Period (B) up to week 52.
(A)

| | Period | | | | | | | | | | | | |
|---|---|---|---|---|---|---|---|---|---|---|---|---|---|
| | Screening | Treatment Period 1 | | | | | | | | | | | |
| | Week | | | | | | | | | | | | |
| | −2 to 0 | 1 | 2 | 3 | 4 | 5 | 6 | 7 | 8 | 9 | 10 | 11 | 12 |
| Informed consenfaasent | X | | | | | | | | | | | | |
| Inclusion/ Exclusion | X | X | | | | | | | | | | | |
| Medical History | X | X | | | | | | | | | | | |
| Pregnancy test | X | X | | | X | | | | X | | | | X |
| Physical examination | X | | | | | | | | | | | | X |
| Weight | X | | X | | X | | X | | X | | X | | X |
| Waist | X | | X | | X | | | | X | | | | X |
| Hip | X | | X | | X | | | | X | | | | X |
| Height | X | | | | | | | | | | | | X |
| Patient photos | X | | | | | | | | | | | | X |
| PHQ-9 & C-SSRS | X | | | | X | | | | X | | | | X |
| REGN4461 IV loading dose | | X | | | | | | | | | | | |
| REGN4461 SC | | X | X | X | X | X | X | X | X | X | X | X | X |
| administrations | | | | | | | | | | | | | |
| Injection site inspection | | X | X | X | X | X | X | X | X | X | X | X | X |
| Vital signs | X | X | X | X | X | X | X | X | X | X | X | X | X |
| ECG (12-lead) | X | X | | | | | | | | | | | X |
| Safety laboratory tests | X | X | X | X | X | X | X | X | X | X | X | X | X |
| Hunger Questionnaire | X | X | X | X | X | X | X | X | X | X | X | X | X |
| Global Hunger Questions | X | X | | | | | | | | | | | X |
| DEXA fat distribution | X | | | | | | | | | | | | X |
| Liver MRI | X | | | | | | | | | | | | X |
| REE | X | | | | | | | | | | | | X |
| SF-36 | X | | | | | | | | | | | | X |
| REGN4461 concentration | | X | X | | X | | X | | X | | X | | X |
| ADA samples | | X | | | | | | | | | | | X |
| Adverse events | X | X | X | X | X | X | X | X | X | X | X | X | X |
| Concomitant meds | X | X | X | X | X | X | X | X | X | X | X | X | X |

(B)

| | Period | | | | | | | | | | | | | | | | | | | |
|---|---|---|---|---|---|---|---|---|---|---|---|---|---|---|---|---|---|---|---|---|
| | Treatment Period 2 | | | | | | | | | | | | | Extended Treatment Period (ongoing) | | | | | | |
| | Week | | | | | | | | | | | | | | | | | | | |
| | 13 | 14 | 15 | 16 | 17 | 18 | 19 | 20 | 21 | 22 | 23 | 24 | 25 | 28 | 32 | 36 | 40 | 44 | 48 | 52 |
| Informed consent/assent | | | | | | | | | | | | | | | | | | | | |
| Inclusion/Exclusion | | | | | | | | | | | | | | | | | | | | |
| Medical history | | | | | | | | | | | | | | | | | | | | |
| Pregnancy test | | | | X | | | | X | | | | | X | X | X | X | X | X | X | X |
| Physical examination | | | | | | | | | | | | | X | | | | | | | X |
| Weight | | X | | X | | X | | X | | X | | | X | X | X | X | X | X | X | X |
| Waist | | | | X | | | | X | | | | | X | X | X | X | X | X | X | X |
| Hip | | | | X | | | | X | | | | | X | X | X | X | X | X | X | X |
| Height | | | | | | | | | | | | | X | | | | | | | X |
| Patient photos | | | | | | | | | | | | | X | | | | | | | X |
| PHQ-9 & C-SSRS | | | | X | | | | X | | | | | X | | X | | X | | X | X |
| REGN4461 SC | X | X | X | X | X | X | X | X | X | X | X | X | X | X | X | X | X | X | X | X |
| administrations | | | | | | | | | | | | | | | | | | | | |
| Injection site inspection | X | X | X | X | X | X | X | X | X | X | X | X | X | X | X | X | X | X | X | X |
| Vital signs | X | X | X | X | X | X | X | X | X | X | X | X | X | X | X | X | X | X | X | X |
| ECG (12-lead) | | | | | | | | | | | | | X | | | | | | | X |
| Safety laboratory tests | X | X | X | X | X | X | X | X | X | X | X | X | X | X | X | X | X | X | X | X |
| Hunger Questionnaire | X | X | X | X | X | X | X | X | X | X | X | X | X | X | X | X | X | X | X | X |
| Global Hunger Questions | | | | | | | | | | | | | X | | | | | | | X |
| DEXA fat distribution | | | | | | | | | | | | | X | | | | | | | X |
| Liver MRI | | | | | | | | | | | | | X | | | | | | | X |
| REE | | | | | | | | | | | | | X | | | | | | | X |
| SF-36 | | | | | | | | | | | | | X | | | | | | | X |
| REGN4461 concentration | | X | | X | | X | | X | | X | | | X | X | X | X | X | X | X | X |
| ADA samples | | | | | | | | | | | | | X | | | | | | | X |
| Adverse events | X | X | X | X | X | X | X | X | X | X | X | X | X | X | X | X | X | X | X | X |
| Concomitant meds | X | X | X | X | X | X | X | X | X | X | X | X | X | X | X | X | X | X | X | X |

The patient underwent two sessions of plasmapheresis on week 1, prior to REGN4461 treatment and at week 2, after initiation of REGN4461 treatment. Thereafter, there was no clinical need for regular plasmapheresis. Starting at week 4, the patient's triglyceride levels decreased relative to baseline and dropped below 500 mg/dL at the week 6 measurement. Thereafter, triglyceride levels that were measured at various times to week 94 averaged about 522 mg/dL. Treatment with REGN4461 was associated with a reduction of triglycerides from 1288 mg/dL at baseline, to 426 mg/dL at week 12 (66.93% decrease), and 231 mg/dL at week 25 (82.07% decrease), in this patient with atypical partial lipodystrophy and neutralizing leptin antibodies. At week 25, the patient met the pre-determined primary endpoint of achieving fasting triglyceride levels <500 mg/dL without the need for ongoing plasmapheresis.

Concomitant with REGN4461 treatment, the patient lost 4.2 kilograms from baseline in the first 8 weeks and maintained a lower body weight for the rest of the observation period (4.5 kg loss from baseline at week 25). Body weight increased by 0.2 kilograms at week 68 and by 0.9 kilograms at week 94.

In parallel with the body weight loss, a reduction in waist circumference from 99 cm to 90.5 cm from baseline to week 25 was observed.

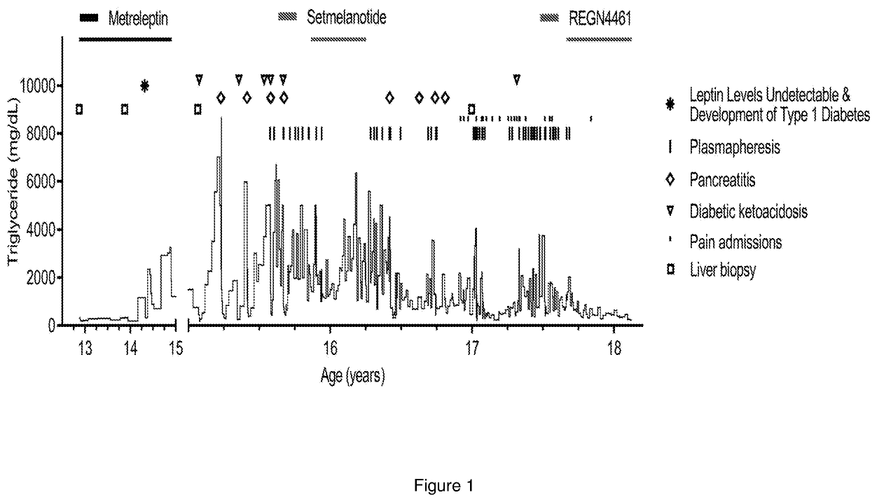

LEPR agonist treatment was also associated with a decrease in hospital admissions for pain and pancreatitis. In the 12 months preceding initiation of LEPR agonist mAb treatment, she had a total of 22 admissions and spent 64 days as an inpatient out of 365 days. After treatment initiation, but for two admissions for SAEs as discussed herein, the patient did not require any in-patient hospitalizations and had only one episode of abdominal pain requiring emergency room evaluation. The patient also discontinued regular opioid medications for pain. FIG. 1 summarizes the early course of the disease over time and her metabolic parameters during different therapeutic interventions.

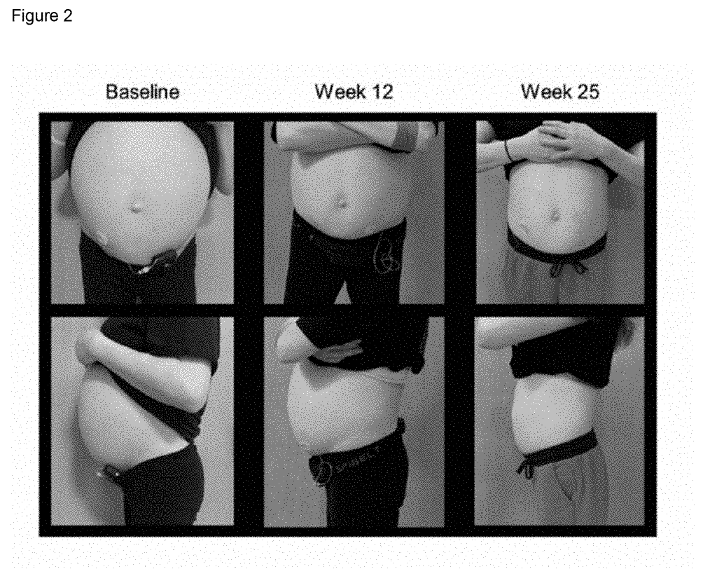

Liver-related parameters were assessed as part of the treatment plan. Hepatic steatosis estimated by magnetic resonance imaging proton density fat fraction (PDFF) decreased from 29.89% (SD: 7.85, ROI:14594 mm²) at baseline to 16.63% (SD: 1.89, ROI: 2807 mm²) at week 12, which further decreased to 12.52% (SD: 2.01, ROI: 2046 mm²) at week 25. Marginal reductions were observed in liver enzymes over time. A reduction in the patient's liver size from baseline was evident by physical examination of liver span (FIG. 2). In accordance, the longitudinal diameter of the liver measured craniocaudally from the uppermost right hemi-diaphragm to the inferior tip of the right lobe decreased from 352 mm at baseline to 294 mm at week 12 and 270 mm at week 25. There was a reduction in hepatic stiffness from 8.62 kPa (SD: 3.09) at week 12 to 6.57 kPa (SD: 1.90) at week 25. For measures of hepatic stiffness, a comparison to baseline status was not possible as hepatic elastography could not be completed due to the patient's severe abdominal pain at baseline leading to an inability to breath-hold and obtain proper positioning of the coil. Liver stiffness remained stable at week 52 (5.9-7.7 kPa). Preliminary hepatic stiffness was 7.11±3.12 at week 79 (unscheduled visit). The patient's spleen size also decreased from 15.8 cm to 14.1 cm at week 12 and 13.5 cm at week 25.

With respect to glycemic control, REGN4461 treatment was associated with a slight reduction in the patient's HbA1c levels from 9.5% at baseline, to 9.1% at week 12, and 9.0% at week 25. HbA1c levels dipped from week 25 to week 79, again rising to about 10% at week 82 and week 84.

Parallel reductions in her average glucose levels and total daily insulin requirement also occurred. Blood glucose at week −2 to 0 was 255 mg/dL and measurements averaged about 122.9 until week 94. Insulin doses were adjusted based on continuous glucose monitoring (CGM) readings. The patient developed hypoglycemia during REGN4461 treatment, which necessitated a reduction in her insulin dose to prevent hypoglycemia events. Overall, however, glycemic control remained suboptimal in this complicated patient with lipodystrophy and type 1 diabetes.

Since leptin replacement in lipodystrophic patients reduces self-reported hunger in the post-prandial state associations of REGN4461, treatment with hunger were explored. Hunger scores showed a slight temporary increase during the first four weeks of the treatment and then returned to baseline levels. Per the Global Hunger Questionnaire, the patient described moderate hunger at baseline and mild hunger and weeks 12 and 25. Subjectively, she felt “much less hungry” at week 12 compared to baseline and “somewhat less hungry” at week 25 compared to previous weeks.

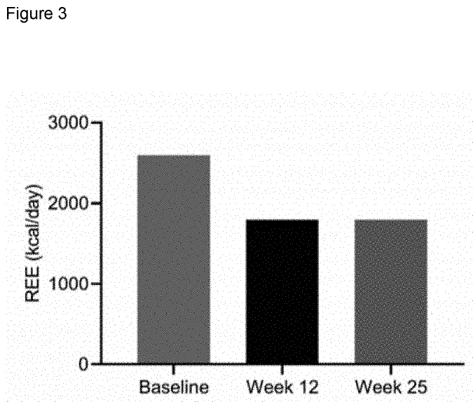

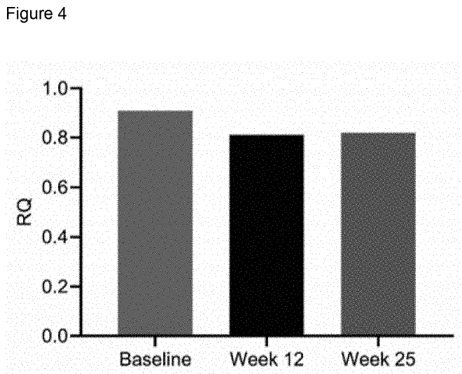

Resting energy expenditure (REE), which represents roughly 60% of total energy expenditure, decreased from 2599 kcal at baseline to 1799 kcal (FIG. 3) at week 25 and remained the same at week 25. It is possible that this could represent a reduction in overall food intake or changes in energy expenditure related to improvement in anxiety and pain. Also, respiratory quotient (RQ) decreased from 0.92 at baseline to 0.81 at week 12 and 0.82 at week 25 (FIG. 4), which may be attributed to a decrease in carbohydrate intake, increase in fatty acid oxidation or reduced de novo lipogenesis.

Because the patient presented with hypogonadotropic hypogonadism, the patient's pubertal development was monitored during treatment with the LEPR agonist, REGN4461. During the initial 25-week treatment period, the patient's tanner stage progressed from IV to V (genital) and III to IV (breast development). At week 25, increases from baseline were observed in her follicle stimulating hormone (FSH; from 1.4 mIU/mL to 3.9 and 4.4 mIU/mL), luteinizing hormone (LH; from 0.2 mIU/mL to 4.8 and 4.4 mIU/mL), estradiol (from <20 pg/mL to 29 and 64 pg/mL), and testosterone (from <0.05 ng/mL to 0.16 and 0.40 ng/mL). At week 25, the patient had not started menstruating. Of note, her pituitary MRI was normal.

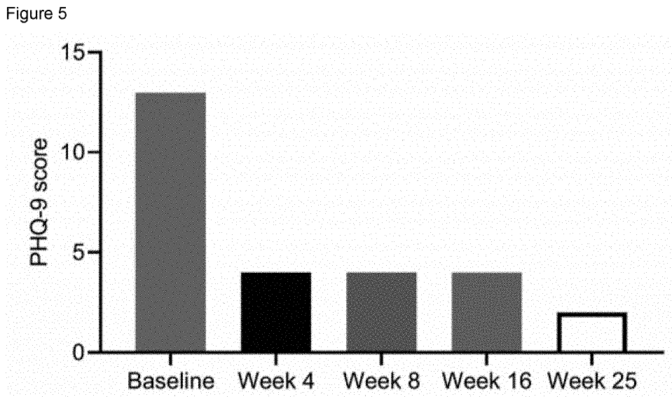

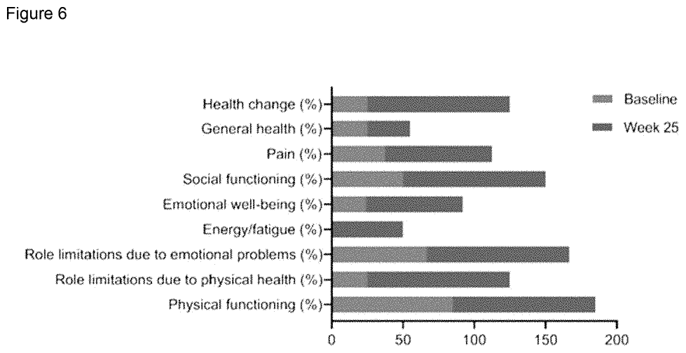

Substantial improvements in the patient's mood and the ability to participate in daily activities were associated with REGN4461 treatment. Subjective indicators of quality of life also showed improvements. The PHQ-9 (Patient Health Questionnaire-9) score, a multipurpose instrument for screening, diagnosing, monitoring and measuring the severity of depression, decreased from 13 at baseline (indicates moderate depression) to 4 at week 4, 4 at week 16, and later to 2 at week 25 (indicates no depression; FIG. 5). Moreover, improvements were observed in all components of SF-36 scores at week 25: physical functioning increased from 85% to 100%, role limitations due to physical health from 25% to 100%, role limitations due to emotional problems from 67% to 100%, energy/fatigue from 0% to 50%, emotional well-being from 24% to 68%, social functioning from 50% to 100%, pain from 37.5% to 75%, general health from 25% to 30%, and health change from 25% to 100% (FIG. 6). Of note, her venlafaxine and buspirone doses were reduced during the therapy from 150 mg to 75 mg and from 15 mg to 2.5 mg, respectively.

The SF-36 Health Questionnaire is a multi-purpose, short-form health survey with only 36 questions. It yields an 8-scale profile of functional health and well-being scores as well as psychometrically-based physical and mental health summary measures and a preference-based health utility index. It is a generic measure, as opposed to one that targets a specific age, disease, or treatment group.

Overall, REGN4461 was well-tolerated. No injection site reactions were reported except minor bleeding from the injection site. Early adverse events included urinary tract infection, upper respiratory infection, abdominal pain, diarrhea, vomiting, nausea, hypoglycemia, and hyperglycemia. These adverse events were considered to be not related to the drug. Two SAEs were recorded. Episodes of hypoglycemia and hyperglycemia may be associated with changes in insulin sensitivity after treatment with REGN4461 and the prescribed dose reductions in exogenous insulin, respectively.

TABLE 1-3
Record of Drug Use for Pain and Depression (First Dose of
REGN4461 Given on Day 0).

| Drug | Indication | Day\hat{ } | Dose |
|---|---|---|---|
| Gabapentin | Pain | | |
| Dose before first | | Day 0 | 3100 mg/day PO |
| REGN4461 dose | | −2 | 2400 mg/day |
| Dose change | | | |
| Dose change | | 6 | 1500 mg/day |
| Dose change | | 7 | 1200 mg/day |
| Oxycodone | Pain | | |
| Dose before first | | | PRN |
| REGN4461 dose | | | |
| Stopped* | | Day 0 | |
| Venlafaxine | Depression | | |
| Dose before first | | day 0 | 150 mg/day PO |
| REGN4461 dose | | | |
| Dose change | | 77 | 75 mg/day |
| Buspirone | Anxiety | | |
| Dose before first | | day 0 | 15 mg/day PO |
| REGN4461 dose | | | |
| Dose change | | −2 | 10 mg/day |
| Dose change | | 70 | 5 mg/day |
| Dose change | | 273 | 10 mg/day |
| Paracetamol | Pain | | |
| Dose before first | | day 0 | PRN |
| REGN4461 dose | | | |

Adverse Events

| Extra doses given/ | | | |
|---|---|---|---|
| drugs used to control | | | |
| pain during episodes | AE report | Day | Dose |
| Oxycodone | SAE reported; | 268 | 15 mg PO |
| | abdominal | | |
| | pain/nausea/vomiting | | |
| Oxycodone | SAE reported; | 269 | 30 mg PO |
| | abdominal | | |
| | pain/nausea/vomiting | | |
| Oxycodone | SAE reported; | 270 | 10 mg PO |
| | abdominal | | |
| | pain/nausea/vomiting | | |
| Gabapentin | SAE reported; | 269 | 1200 mg PO (two |
| | abdominal | | doses of 600 mg) |
| | pain/nausea/vomiting | | |
| Buspirone | SAE reported; | 268 | 10 mg PO |
| | abdominal | | |
| | pain/nausea/vomiting | | |
| Buspirone | SAE reported; | 269 | 10 mg PO |
| | abdominal | | |
| | pain/nausea/vomiting | | |
| Paracetamol | AE reported; | 69 | 500 mg PO |
| | abdominal | | |
| | pain/nausea | | |
| Paracetamol | AE reported; | 117 | 500 mg PO |
| | abdominal | | |
| | pain/vomiting/diarrhea | | |
| Paracetamol | AE reported; | 118 | 500 mg PO |
| | abdominal | | |
| | pain/vomiting/diarrhea | | |
| Paracetamol | AE reported; | 119 | 1000 mg PO |
| | abdominal | | |
| | pain/vomiting/diarrhea | | |
| Paracetamol | No AE report in file; | 130 | 650 mg PO |
| | abdominal pain | | |
| Ketamin | AE reported; | −2 | 25 mg IV followed by |
| | abdominal pain | | 0.2 mg/kg/hr infusion |
| Ketorolac | SAE reported; | 269 | 15 mg IV |
| | abdominal | | |
| | pain/nausea/vomiting | | |
| Lorazepam | SAE reported; | 269 | 0.5 mg PO |
| | abdominal | | |
| | pain/nausea/vomiting | | |
| Morphine | SAE reported; | 270 | 12 mg total (three |
| | abdominal | | doses of 4 mg) |
| | pain/nausea/vomiting | | |

\hat{ }Measured from date of first IV dose (day 0)
* Not formally discontinued by treating physician, but no further regular dosing was required. Additional instances of oxycodone usage by the patient were in connection with a kidney/liver biopsy and port removal and a urinary tract infection.
SAE = serious adverse event
PO = per os
PRN = pro re nata or as-needed
PRN oxycodone use (prior to the initiation of REGN4461) was at least 2 to 3 doses for at least 3 days of the week for at least 12 months.

A summary of adverse events (AEs) during the period of day 339-day 704 (from and including the date of the first SC dose-to and including the AE date) is as follow:
Non-serious adverse event: Day 432, sinus infection
Non-serious adverse event: Day 507, urinary tract infection
Serious adverse event (SAE): On day 536, the patient began feeling abdominal pain, which worsened over the next day and led her to the local emergency department the next day. Lab work showed elevated triglycerides of 1103 mg/dL, as well as AST of 58 IU/L and ALT of 61 IU/L. CT scan of her abdomen and pelvis with contrast were obtained and revealed no acute pancreatitis. Patient was given oxycodone, IV Toradol, and IV ketamine for pain control. Patient was admitted for the concern of hypertriglyceridemia and abdominal pain, and placed on IV insulin drip to lower triglycerides. Repeat triglycerides the evening of day 537 were 586 mg/dL and 477 mg/dL the morning of day 538. The patient was given two dosed of IV morphine for pain control on day 538 Her condition continued to improved and she was discharged on day 539.
Serious adverse event (SAE): On day 628 when the patient began feeling abdominal pain and associated nausea, which worsened over the next day and led her to the local emergency department on day 629. Lab work showed slightly elevated triglycerides of 332 mg/dL, AST of 40 IU/L, and ALT of 55 IU/L. Her lipase level on day 629 was 13 and within normal limits. Patient was given oxycodone, IV Toradol, and IV ketamine for pain control. She was admitted for the concern of hypertriglyceridemia and abdominal pain. An abdominal ultrasound on day 630 showed a normal pancreas, and an x-ray on day 629 showed bowel gas pattern concerning for ileus or gastroenteritis and large fecal load. Repeat triglycerides the evening of day 630 were 475 mg/dL. The patient still reported severe abdominal pain and was given Roxicodone for pain control and Toradol for inflammation control. The patient's condition improved and she was discharged on day 632. The patient was then seen by study doctor in clinic on day 635, at which time the abdominal pain had completely resolved.

Both SAEs were to be expected due to the patient's history and unrelated to REGN4461.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants to relate to each and every individual publication, database entry (e.g., Genbank sequences or GeneID entries), patent application, or patent, each of which is clearly identified in even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

1. A method for reducing or preventing pain, anxiety and/or depression, in a patient in need thereof, comprising administering, to the patient, an effective amount of LEPR agonist.

2. The method of claim 1 wherein the patient achieves one or more of:
a reduction in pain,
a reduction in the use of analgesics,
a reduction in the use of anxiolytics,
a reduction in the use of anti-depressants,
a reduction in analgesic seeking behavior,
a reduction in pro re nata use of analgesics,
a reduction in incidence of analgesic overdose, and/or
a reduction in the incidence of death due to abuse of analgesics.

3. The method of claim 2 wherein the analgesic is oxycodone.

4. The method of claim 1 wherein the pain, anxiety and/or depression is associated with a leptin deficiency or leptin resistance condition.

5. A method for reducing or maintaining a reduction in:
pain,
use of analgesics,
use of anxiolytics,
use of anti-depressants,
analgesic seeking behavior,
pro re nata use of analgesics,
analgesic overdose, and/or
the incidence of death due to abuse of analgesics;
in a patient in need thereof suffering from pain and/or a leptin deficiency or leptin resistance condition comprising administering, to the patient, an effective amount of LEPR agonist.

6. The method of claim 2 wherein said reduction is within less than 1, 1, 2, 3, 4, or 5 days of the first administration or second administration of the LEPR agonist.

7. The method of claim 1 wherein the pain is generalized pain, abdominal pain, renal pain, liver pain and/or pain due to hepatomegaly, liver stiffness and/or pancreatitis.

8. The method of claim 7 wherein the abdominal pain is accompanied by nausea and/or vomiting.

9. The method of claim 1 wherein the pain is neuropathy pain, arthritis pain, chronic back pain, fibromyalgia pain, myopathy pain, central pain, chronic central pain, pain caused by centralization and/or hypersensitization of pain by the central nervous system and/or central pain syndrome pain.

10. The method of claim 1 wherein the use of analgesics, anxiolytics and/or anti-depressants is reduced concomitantly with or prior to the first administration of the LEPR agonist.

11. The method of claim 1 wherein the patient continues episodic use of analgesics.

12. The method of claim 1 wherein said pain, use of analgesics, anxiolytics and/or anti-depressants is chronic.

13. The method of claim 1 wherein a reduction in pain, anxiety and/or depression is as measured by PHQ-9 and/or SF-36 score.

14. The method of claim 2 wherein a reduction in pro re nata use of analgesics does not include episodic use of analgesics.

15. The method of claim 14 wherein episodic use of analgesics comprises use of analgesics for up to 5, 6, 7 or 8 days.

16. The method of claim 2 wherein the analgesic is an opioid, a non-opioid, a calcitonin gene-related peptide (CGRP) inhibitor, a cyclooxygenase-2 inhibitor, a gepant, an anti-CGRP monoclonal antibody, a nonsteroidal anti-inflammatory drug, a salicylate, acetaminophen, acetylsalicylic acid, alfentanil, aspirin & citric acid & sodium bicarbonate, bromfenac, celecoxib, choline salicylate & magnesium salicylate, codeine, concentrate of poppy straw, dextromoramide, dextropropoxyphene, diclofenac, diclofenac & misoprostol, diflunisal, diflunisal, dihydrocodeine, dihydroetorphine, diphenoxylate, eptinezumab, erenumab, esomeprazole & naproxen, ethylmorphine, etodolac, etorphine, famotidine & ibuprofen, fenoprofen, fentanyl, flurbiprofen, fremanezumab, gabapentin, galcanezumab, heroin, hydrocodone, hydromorphone, ibuprofen, indomethacin, ketamin, ketobemidone, ketoprofen, ketorolac, levorphanol, magnesium salicylate, meclofenamat, mefenamic acid, meloxicam, methadone, methadone, morphine, morphine-n-oxide, nabumetone, naproxen, nicomorphine, norcodeine, opium, oripavine, oxaprozin, oxycodone, oxymorphone, pethidine, pethidine intermediate, phenylbutazone, pholcodine, piritramide, piroxicam, remifentanil, rimegepant sulfate, salsalate, sufentanil, sulindac, thebaine or tilidine, tolmetin, ubrogepant or valdecoxib.

17. The method of claim 16 wherein the analgesic is an opioid.

18. The method of claim 16 wherein the analgesic is not a non-opioid.

19. The method of claim 2 wherein the anxiolytic is a benzodiazepine, a tricyclic antidepressant, alprazolam, alprazolam, an agonist of melatonin receptor, an anesthetic, an antihistamine, an SNRI, an SSRI, buspirone, clonazepam, diazepam, estazolam, eszopiclone, flurazepam, lorazepam, quazepam, temazepam, triazolam, zaleplon, zolpidem or zopiclone.

20. The method of claim 2 wherein the anti-depressant is a monoamine oxidase inhibitor, a selective serotonin reuptake inhibitors (SSRI), a serotonin and norepinephrine reuptake inhibitors (SNRI), a tricyclic antidepressant, amitriptyline, an atypical antidepressant, bupropion, citalopram, desipramine, desvenlafaxine and levomilnacipran, doxepin, duloxetine, escitalopram, fluoxetine, imipramine, isocarboxazid, mirtazapine, nortriptyline, paroxetine, phenelzine, selegiline, sertraline, tranylcypromine, trazodone, venlafaxine, vilazodone or vortioxetine.

21. A method of reducing hospitalization or emergency medical intervention of a patient due to pain and/or of a patient suffering from a leptin deficiency or leptin resistance condition due to pain, anxiety and/or depression comprising administering, to the patient in need thereof, an effective amount of LEPR agonist.

22. The method of claim 21 wherein the leptin deficiency or leptin resistance condition is monogenic obesity, obesity, metabolic syndrome, diet-induced food craving, functional hypothalamic amenorrhea, type 1 diabetes, type 2 diabetes, insulin resistance, possession of neutralizing anti-leptin autoantibodies, severe insulin resistance including severe insulin resistance due to mutation in insulin receptor, severe insulin resistance not caused by mutation in the insulin receptor, severe insulin resistance caused by a mutation in downstream signaling pathways or induced by other causes, non-alcoholic and alcoholic fatty liver diseases, Alzheimer's disease, leptin deficiency, leptin resistance, a lipodystrophy, Leprechaunism/Donohue syndrome or Rabson-Mendenhall syndrome.

23. The method of claim 21 wherein the leptin deficiency or leptin resistance condition is a lipodystrophy which is congenital generalized lipodystrophy, acquired generalized lipodystrophy, familial partial lipodystrophy, acquired partial lipodystrophy, centrifugal abdominal lipodystrophy, lipoatrophia annularis, localized lipodystrophy, and HIV-associated lipodystrophy.

24. The method of claim 21 wherein the LEPR agonist is an isolated agonist antibody or antigen-binding fragment that binds specifically to LEPR.

25. The method of claim 21 wherein the LEPR agonist is an isolated agonist antibody or antigen-binding fragment that binds specifically to LEPR comprising:
(i) a light chain variable region that comprises the LCDR1, LCDR2 and LCDR3 of a light chain variable region that comprises the amino acid sequence set forth in SEQ ID NO: 10; and
a heavy chain variable region that comprises the HCDR1, HCDR2 and HCDR3 of a heavy chain variable region that comprises the amino acid sequence set forth in SEQ ID NO: 2;
(ii) a light chain variable region that comprises the LCDR1, LCDR2 and LCDR3 of a light chain variable region that comprises the amino acid sequence set forth in SEQ ID NO: 10; and
a heavy chain variable region that comprises the HCDR1, HCDR2 and HCDR3 of a heavy chain variable region that comprises the amino acid sequence set forth in SEQ ID NO: 18;
(iii) a light chain variable region that comprises the LCDR1, LCDR2 and LCDR3 of a light chain variable region that comprises the amino acid sequence set forth in SEQ ID NO: 10; and
a heavy chain variable region that comprises the HCDR1, HCDR2 and HCDR3 of a heavy chain variable region that comprises the amino acid sequence set forth in SEQ ID NO: 26;
(iv) a light chain variable region that comprises the LCDR1, LCDR2 and LCDR3 of a light chain variable region that comprises the amino acid sequence set forth in SEQ ID NO: 10; and
a heavy chain variable region that comprises the HCDR1, HCDR2 and HCDR3 of a heavy chain variable region that comprises the amino acid sequence set forth in SEQ ID NO: 34;
(v) a light chain variable region that comprises the LCDR1, LCDR2 and LCDR3 of a light chain variable region that comprises the amino acid sequence set forth in SEQ ID NO: 10; and
a heavy chain variable region that comprises the HCDR1, HCDR2 and HCDR3 of a heavy chain variable region that comprises the amino acid sequence set forth in SEQ ID NO: 42;
(vi) a light chain variable region that comprises the LCDR1, LCDR2 and LCDR3 of a light chain variable region that comprises the amino acid sequence set forth in SEQ ID NO: 10; and
a heavy chain variable region that comprises the HCDR1, HCDR2 and HCDR3 of a heavy chain variable region that comprises the amino acid sequence set forth in SEQ ID NO: 50;
(vii) a light chain variable region that comprises the LCDR1, LCDR2 and LCDR3 of a light chain variable region that comprises the amino acid sequence set forth in SEQ ID NO: 10; and
a heavy chain variable region that comprises the HCDR1, HCDR2 and HCDR3 of a heavy chain variable region that comprises the amino acid sequence set forth in SEQ ID NO: 58;
(viii) a light chain variable region that comprises the LCDR1, LCDR2 and LCDR3 of a light chain variable region that comprises the amino acid sequence set forth in SEQ ID NO: 10; and
a heavy chain variable region that comprises the HCDR1, HCDR2 and HCDR3 of a heavy chain variable region that comprises the amino acid sequence set forth in SEQ ID NO: 66;
(ix) a light chain variable region that comprises the LCDR1, LCDR2 and LCDR3 of a light chain variable region that comprises the amino acid sequence set forth in SEQ ID NO: 10; and
a heavy chain variable region that comprises the HCDR1, HCDR2 and HCDR3 of a heavy chain variable region that comprises the amino acid sequence set forth in SEQ ID NO: 74;
(x) a light chain variable region that comprises the LCDR1, LCDR2 and LCDR3 of a light chain variable region that comprises the amino acid sequence set forth in SEQ ID NO: 90; and
a heavy chain variable region that comprises the HCDR1, HCDR2 and HCDR3 of a heavy chain variable region that comprises the amino acid sequence set forth in SEQ ID NO: 82;
(xi) a light chain variable region that comprises the LCDR1, LCDR2 and LCDR3 of a light chain variable region that comprises the amino acid sequence set forth in SEQ ID NO: 90; and
a heavy chain variable region that comprises the HCDR1, HCDR2 and HCDR3 of a heavy chain variable region that comprises the amino acid sequence set forth in SEQ ID NO: 98; or
(xii) a light chain variable region that comprises the LCDR1, LCDR2 and LCDR3 of a light chain variable region that comprises the amino acid sequence set forth in SEQ ID NO: 90; and
a heavy chain variable region that comprises the HCDR1, HCDR2 and HCDR3 of a heavy chain variable region that comprises the amino acid sequence set forth in SEQ ID NO: 106; or
an antibody or antigen-binding fragment that binds to the same epitope as any one or more of (i)-(xii) and/or competes for binding to LEPR with any one or more of (i)-(xii).

26. The method of claim 21 wherein the LEPR agonist is an isolated agonist antibody or antigen-binding fragment that binds specifically to LEPR comprising:
(i) a light chain variable region that comprises the amino acid sequence set forth in SEQ ID NO: 10; and
a heavy chain variable region that comprises the amino acid sequence set forth in SEQ ID NO: 2;
(ii) a light chain variable region that comprises the amino acid sequence set forth in SEQ ID NO: 10; and
a heavy chain variable region that comprises the amino acid sequence set forth in SEQ ID NO: 18;
(iii) a light chain variable region that comprises the amino acid sequence set forth in SEQ ID NO: 10; and
a heavy chain variable region that comprises the amino acid sequence set forth in SEQ ID NO: 26;
(iv) a light chain variable region that comprises the amino acid sequence set forth in SEQ ID NO: 10; and
a heavy chain variable region that comprises the amino acid sequence set forth in SEQ ID NO: 34;
(v) a light chain variable region that comprises the amino acid sequence set forth in SEQ ID NO: 10; and
a heavy chain variable region that comprises the amino acid sequence set forth in SEQ ID NO: 42;
(vi) a light chain variable region that comprises the amino acid sequence set forth in SEQ ID NO: 10; and
a heavy chain variable region that comprises the amino acid sequence set forth in SEQ ID NO: 50;
(vii) a light chain variable region that comprises the amino acid sequence set forth in SEQ ID NO: 10; and
a heavy chain variable region that comprises the amino acid sequence set forth in SEQ ID NO: 58;
(viii) a light chain variable region that comprises the amino acid sequence set forth in SEQ ID NO: 10; and
a heavy chain variable region that comprises the amino acid sequence set forth in SEQ ID NO: 66;
(ix) a light chain variable region that comprises the amino acid sequence set forth in SEQ ID NO: 10; and
a heavy chain variable region that comprises the amino acid sequence set forth in SEQ ID NO: 74;
(x) a light chain variable region that comprises the amino acid sequence set forth in SEQ ID NO: 90; and
a heavy chain variable region that comprises the amino acid sequence set forth in SEQ ID NO: 82;
(xi) a light chain variable region that comprises the amino acid sequence set forth in SEQ ID NO: 90; and
a heavy chain variable region that comprises the amino acid sequence set forth in SEQ ID NO: 98; or
(xii) a light chain variable region that comprises the amino acid sequence set forth in SEQ ID NO: 90; and
a heavy chain variable region that comprises the amino acid sequence set forth in SEQ ID NO: 106; or
an antibody or antigen-binding fragment that binds to the same epitope as any one or more of (i)-(xii) and/or competes for binding to LEPR with any one or more of (i)-(xii).

27. The method of claim 21 wherein the LEPR agonist is mibavademab.

28. The method of claim 21 wherein the effective amount of LEPR agonist is one or more intravenous doses of about 5 mg/kg and one or more subcutaneous doses of about 250-450 mg once weekly thereafter.

29. The method of claim 28 wherein 250-450 mg is 300 or 450 mg.

30. The method of claim 28 wherein the effective amount of LEPR agonist is one intravenous dose of about 5 mg/kg and one or more subcutaneous doses of about 300 mg or 450 mg once weekly thereafter.

31. The method of claim 21 further comprising administering a further therapeutic agent to the patient.

32. The method of claim 31 wherein the further chemotherapeutic agent is human leptin, metreleptin, a PCSK9 inhibitor, an anti-PCSK9 antibody, alirocumab, evolocumab, bococizumab, lodelcizumab, ralpancizumab, an HMG-CoA reductase inhibitor, atorvastatin, rosuvastatin, cerivastatin, pitavastatin, fluvastatin, simvastatin, lovastatin, pravastatin, ezetimibe, insulin, an insulin variant, an insulin secretagogue, metformin, a sulfonylurea, a sodium glucose cotransporter 2 (SGLT2) inhibitor, dapaglifozin, canaglifozin, empagliflozin, a selective agonist of the MC₄ receptor, setmelanotide, a GLP-1 agonist or analogue, extendin-4, exenatide, liraglutide, lixisenatide, albiglutide, dulaglutide, a glucagon (GCG) inhibitor, an anti-GCG antibody, a glucagon receptor (GCGR) inhibitor, an anti-GCGR antibody, a small molecule GCGR antagonist, a GCGR-specific antisense oligonucleotide, an anti-GCGR aptamer, an angiopoietin-like protein (ANGPTL) inhibitor, an anti-ANGPTL3 antibody, an anti-ANGPTL4 antibody, an anti-ANGPTL8 antibody, phentermine, orlistat, topiramate, bupropion, topiramate and phentermine, bupropion and naltrexone, bupropion and zonisamide, pramlintide and metrelepin, lorcaserin, cetilistat, tesofensine and/or velneperit.

33. The method of claim 21 wherein the patient further achieves one or more of:
a reduction in body weight,
a reduction in food intake,
a reduction in adiposity,
a reduction in hepatic stiffness
improved glycemic control,
improved insulin sensitivity,
an improvement in dyslipidemia,
an improvement in hepatic steatosis,
an improvement in hepatomegaly,
a reduction in pancreatitis,
reduced serum triglyceride levels,
a reduction in the frequency of plasmapheresis,
reduced total cholesterol levels, and/or
reduced serum LDL-C levels.

34. The method of claim 21 wherein the patient suffers from:
obesity,
hyperphagia,
excess adiposity,
hepatic stiffness,
low glycemic control,
diabetes,
insulin resistance,
dyslipidemia,
hepatic steatosis,
hepatomegaly,
pancreatitis,
elevated serum triglyceride levels,
elevated total cholesterol levels, and/or
elevated serum LDL-C levels;
and/or receives regular plasmapheresis.
